(19) United States
(12) Patent Application Publication
(10) Pub. No.: US 2025/0241676 A1
(43) Pub. Date: Jul. 31, 2025

(54) MEDICAL APPARATUS GUIDANCE SYSTEMS AND METHODS

(71) Applicants: Evanesce Medical, Inc., Excelsior, MN (US); Mayo Foundation for Medical Education and Research, Rochester, MN (US)

(72) Inventors: Patrick Jan BOLDENOW, Bloomington, MN (US); Lawrence WALES, Maplewood, MN (US); Steven R. CLENDENEN, Ponte Vedra Beach, FL (US); Jeffrey PETERS, Excelsior, MN (US); Ishmael BENTLEY, Edina, MN (US); Cole CHEN, Plymouth, MN (US)

(73) Assignees: Evanesce Medical, Inc., Excelsior, MN (US); Mayo Foundation for Medical Education and Research, Rochester, MN (US)

(21) Appl. No.: 19/036,778

(22) Filed: Jan. 24, 2025

Related U.S. Application Data
(60) Provisional application No. 63/573,339, filed on Apr. 2, 2024.
(60) Provisional application No. 63/625,816, filed on Jan. 26, 2024.

(51) Int. Cl. A61B 17/34

(52) U.S. Cl. CPC A61B 17/3403; A61B 2017/3407; A61B 2017/3411

(57) ABSTRACT

A guidance device is provided. The guidance device may include a fastener, a panel including a first surface, a second surface opposite the first surface, and a plurality of holes formed on the first surface and configured to receive the fastener. The plurality of holes may include a first hole and a second hole. The fastener may be configured to pass through the first hole and the second hole, and form a loop. The loop formed by the fastener may be configured to receive a medical device between the fastener and the first surface of the panel. A size of the loop may be adjustable to receive the medical device and secure the medical device.

### CROSS-REFERENCE TO RELATED APPLICATION

This application claims the benefit of U.S. Provisional Patent Application 63/625,816, filed Jan. 26, 2024, and U.S. Provisional Patent Application 63/573,339, filed Apr. 2, 2024, the entire contents of each of which are incorporated herein by reference.

### FIELD

The present disclosure relates generally to devices, systems, and methods for medical apparatus guidance. More specifically, the present disclosure is concerned with a guidance device to assist in guidance of a medical apparatus, such as a needle and catheter.

### INTRODUCTION

In medical practices, particularly those involving the insertion of devices into a patient, achieving precision and maintaining stability are areas where continuous improvement is desired for successful outcomes. Traditional methods often rely on manual unguided insertion and stabilization of these medical devices, which can be both challenging and imprecise. Additionally, the requirement to adjust the angle of entry while ensuring consistent targeting of a specific point on the patient adds further complexity to these procedures, often necessitating skilled manual manipulation and, in some cases, additional personnel. Traditional needle guides also often attach to components such that the attachment causes obstruction of certain views (e.g., of an insertion site), resulting in a greater likelihood of insertion error.

This introduction section is provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the disclosure, methods, devices, and systems are disclosed for assisting in the guidance of a medical apparatus, such as a needle and catheter.

According to aspects disclosed herein, a guidance device for the guidance of a medical apparatus is provided. In some embodiments, the guidance device may include a fastener, a panel including a first surface, a second surface opposite the first surface, and a plurality of holes formed on the first surface and configured to receive the fastener. The plurality of holes may include a first hole and a second hole. The fastener may be configured to pass through the first hole and the second hole, and form a loop. The loop formed by the fastener may be configured to receive a medical device between the fastener and the first surface of the panel. A size of the loop may be adjustable to receive the medical device and secure the medical device.

According to other aspects disclosed herein, a guidance device may include one or more fasteners and a panel including a first surface, a second surface opposite the first surface, and a plurality of holes formed on the first surface and configured to receive the fastener. The plurality of holes may include a first hole, a second hole, a third hole, and a fourth hole. The one or more fasteners may include a first fastener configured to pass through the first hole and the second hole, and form a first loop, and a second fastener configured to pass through the third hole and the fourth hole, and form a second loop. The first loop formed by the first fastener may be configured to receive a first device, and the second loop formed by the second fastener is configured to receive the first device. A size of the first loop and a size of the second loop are adjustable to receive the first device and secure the first device.

According to other aspects disclosed herein, a guidance device may include a first panel, the first panel including: an attachment element on at least a portion of a bottom surface of the first panel, one or more first engagement elements located on a top surface of the first panel, and one or more friction members disposed along the first panel, and a second panel movably connected to the first panel via one or more hinges, the second panel including a guidance element positioned along a the second panel for receiving a medical device, and one or more second engagement elements for engagement with the one or more first engagement elements of the first panel. The first panel and the second panel may define a target region, when aligned, and the guidance device may be configurable between an unlocked configuration, where the first panel and the second panel are movable relative to each other, and a locked configuration, where the first panel and the second panel are secured relative to each other by engagement of the one or more first engagement elements with the one or more second engagement elements.

The above summary is not intended to describe each and every embodiment or implementation of the present disclosure.

### BRIEF DESCRIPTION OF THE DRAWINGS

The accompanying drawings, which are incorporated into and constitute a part of this specification, illustrate various exemplary embodiments and, together with the description, serve to explain the principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

The drawings illustrate example embodiments of the present disclosure and, along with the description, serve to explain the principles of the disclosure. The drawings are only illustrative of certain embodiments and do not limit the disclosure.

FIG. 1 is an isometric view of a guidance device according to some embodiments of the disclosure, shown in an unlocked configuration, according to some embodiments of the disclosure.

FIG. 2 is an isometric view of the guidance device of FIG. 1, shown in a hinged position, according to some embodiments of the disclosure.

FIG. 3 is an isometric view of the guidance device of FIG. 2, shown with a medical device inserted into a guidance element of the guidance device.

FIG. 4 is an isometric view of the guidance device and medical device of FIG. 3, according to some embodiments of the disclosure.

FIG. 5 is an isometric view of the guidance device and medical device of FIG. 3, with a portion of the medical device moved in a first direction, according to some embodiments of the disclosure.

FIG. 6 is an isometric view of the guidance device and medical device of FIG. 3, with a portion of the medical device moved in a second direction, according to some embodiments of the disclosure.

FIG. 7 is an isometric view of the guidance device and medical device of FIG. 3, the guidance device shown in a locked configuration, according to some embodiments of the disclosure.

FIG. 8 is a side section view of the guidance device and medical device of FIG. 7, according to some embodiments of the disclosure.

FIG. 9 is an isometric view of the guidance device according to some embodiments, depicting a panel in an open configuration, according to some embodiments of the disclosure.

FIG. 10 is an isometric view of the guidance device of FIG. 9, illustrating the removal of a catheter from a guidance element, according to some embodiments of the disclosure.

FIG. 11 is an isometric view of the guidance device of FIG. 9, showing the third panel in a closed position with a catheter located within the guidance pathway formed by guidance elements of one or more panels, according to some embodiments of the disclosure.

FIG. 12 is an isometric view of the guidance device of FIG. 11, depicting the device in an unlocked configuration, according to some embodiments of the disclosure.

FIG. 13 is an isometric view of a guidance device, according to some embodiments of the disclosure.

FIG. 14 is an isometric view of the guidance device of FIG. 13, shown with a medical apparatus, according to some embodiments of the disclosure.

FIG. 15 is another isometric view of the guidance device of FIG. 14, according to some embodiments of the disclosure.

FIG. 16 is another isometric view of the guidance device of FIG. 14, according to some embodiments of the disclosure.

FIG. 17 is another isometric view of the guidance device of FIG. 14, according to some embodiments of the disclosure.

FIG. 18A is another isometric view of the guidance device of FIG. 14, according to some embodiments of the disclosure.

FIG. 18B is an isometric view of the guidance device of FIG. 18A, shown in a locked configuration, according to some embodiments of the disclosure.

FIGS. 18C-18J show various views of the guidance device of FIG. 18A, according to some embodiments of the disclosure.

FIGS. 19A-19M show various views of a guidance device, according to some embodiments of the disclosure.

FIG. 20 is an isometric view of a guidance device, according to some embodiments of the disclosure.

FIG. 21 is an isometric view of a guidance device, according to some embodiments of the disclosure.

FIG. 22A is an isometric view of a guidance device, according to some embodiments of the disclosure.

FIG. 22B is a bottom view of the guidance device of FIG. 22A.

FIG. 23 is an isometric view of a guidance device, according to some embodiments of the disclosure.

FIG. 24 is an isometric view of a guidance device, according to some embodiments of the disclosure.

FIG. 25 is an exploded view of the guidance device of FIG. 24.

FIG. 26 illustrates the guidance device of FIG. 24 in a locked configuration.

FIG. 27A is a side view of the guidance device of FIG. 24 in an unlocked configuration.

FIG. 27B is a cross-sectional view of the guidance device along the line A-A of FIG. 26 when the guidance device is in an unlocked configuration.

FIG. 28A is a side view of the guidance device of FIG. 24 in a locked configuration.

FIG. 28B is a cross-sectional view of the guidance device along the line A-A of FIG. 26 when the guidance device is in a locked configuration.

FIG. 29A is a view of a top surface of a second panel of the guidance device of FIG. 24.

FIG. 29B is a view of a bottom surface of the second panel of the guidance device of FIG. 24.

FIG. 30 is a view of a top surface of a first panel of the guidance device of FIG. 24.

FIG. 31 is an isometric view of the guidance device of FIG. 24 with an attachment base.

FIG. 32 is an isometric view of the guidance device of FIG. 31 with a medical device inserted into a guiding member of the guidance device.

While embodiments of the disclosure are amenable to various modifications and alternative forms, specifics thereof have been shown by way of example in the drawings and will be described in some detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

There are many embodiments described and illustrated herein. The described embodiments are neither limited to any single aspect nor implementation thereof, nor to any combinations and/or permutations of such aspects and/or implementations. Moreover, each of the aspects of the described embodiments, and/or implementations thereof, may be employed alone or in combination with one or more of the other aspects of the described embodiments and/or implementations thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

The term “distal end,” or any variation thereof, refers to the portion of a device farthest from an operator of the device during a procedure. Conversely, the term “proximal end,” or any variation thereof, refers to the portion of the device closest to the operator of the device. Further, any use of the terms “around,” “about,” “substantially,” and “approximately” generally mean+/−10% of the indicated value(s).

Systems and methods disclosed herein provide a guidance device that not only facilitates precise and stable positioning of medical devices but also enhances ease of use, particularly in allowing adjustments to the angle of entry while maintaining accurate targeting. An advantage of such a device is its ability to be operated with one hand, freeing the other hand for concurrent tasks, which may be especially beneficial where an additional device may help in further informing accurate entry angles for the medical device.

The present disclosure details a guidance device designed for manual or automated placement of a catheter. It includes two main parts: a first panel, which may adhere (e.g., stick) to the patient's skin, and a second panel, which may move and adjust angles (e.g., angles of insertion of a needle and/or catheter). These panels are connected by one or more hinges and work together to accurately guide a needle and/or catheter to a specific point on the patient's body. It will be understood that the one or more hinges may be living hinges. It will also be understood that another connecting mechanism other than a hinge may be used to connect such panels. A catheter may be inserted into a guidance element in the second panel and guided to a desired target angle, at which point it is inserted into the patient via a needle that is also inserted at the target angle using the guidance device. Once inserted, the second panel rotates to meet the first panel and locks into place, securing the catheter in a friction-fit to ensure the catheter is locked into place and does not make undesirable movements. The guidance element may be cylindrical or may be any other applicable shape such as a cross-sectional rectangular shape, triangular shape, and/or the like to facilitate guidance of a catheter (e.g., a cylindrical catheter). According to embodiments, a thickness of the second panel may be greater than or equal to one or more dimensions of the guidance element such that the guidance element is wholly contained within the wall thickness of the second panel. According to such embodiments, the guidance element may not protrude beyond an external surface of the second panel.

One or more benefits of the guidance device are that it provides stability during the insertion process and offers continued support to the catheter post-insertion, minimizing the risk of displacement or movement. Furthermore, the device's design allows for operation with just one hand, affording the practitioner more flexibility and efficiency. This one-handed operation is particularly beneficial in scenarios where the practitioner needs to manage other tasks or equipment concurrently. This further allows enhanced precision in catheter placement, as the practitioner may utilizes other devices, such as an ultrasounds, to help precisely identify a preferred angle of insertion.

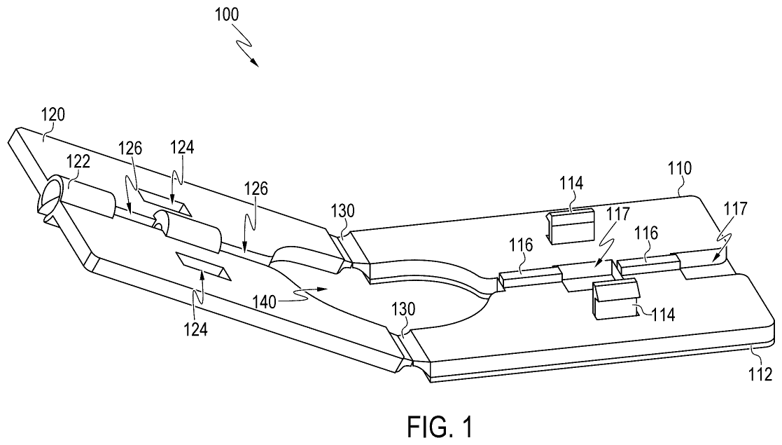

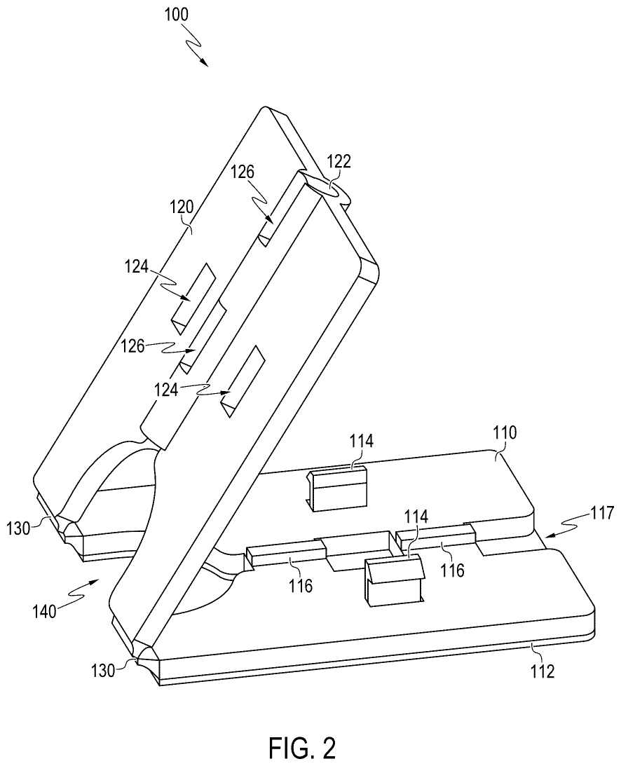

Referring to FIGS. 1 and 2, a guidance device 100 is provided. The guidance device 100 may be used to help guide a medical apparatus (e.g., a medical device 200, such as a needle 210 or a catheter 220) to assist with the targeting and placement of a portion of the medical apparatus. The guidance device 100, generally may include a first panel 110 and a second panel 120. The first panel 110 and the second panel 120 may be connected by one or more hinges 130, and may each include a portion of a cutout defining a target region 140. The hinges 130 may be positioned on opposing sides of the target region 140, such that the first panel 110, second panel 120, and opposing hinges 130 define at least a portion of target region 140. The guidance device 100 is operable to position a medical apparatus relative to a portion of the target region 140 and adjust one or more angle of the medical apparatus by rotating the second panel 120 relative to the first panel 110 about the hinges 130 to position the medical apparatus at a desired angle.

To further assist with alignment of the medical apparatus to the target region 140, one of the first panel 110 or second panel 120 may be attached to a first surface, such as being secured (e.g., via an adhesive) to the skin of a patient. The guidance device 100 is generally configured in either an unlocked configuration or a locked configuration, and is movable between the two configurations. In the unlocked configuration, the guidance device 100 is configured so that first panel 110 and second panel 120 are connected to and/or in contact with one another only by way of hinges 130, and second panel 120 is free to rotate relative to first panel 110. In the locked configuration, the second panel 120 is positioned so one or more surface and/or element of the second panel engages and/or interacts with one or more surface and/or element of the first panel 110 such that the first panel 110 and second panel 120 are locked in place relative to one another and unable to rotate relative to one another about hinges 130.

According to an embodiment, to further assist with alignment of the medical apparatus to the target region 140, guidance device 100 may be configured in an angled intermediate position for insertion of the needle and/or catheter. For example, guidance device 100 may be temporarily locked at a pre-selected, dynamically selected, or user selected angle when in the intermediate position. Such an angle may be formed between the first panel 110 and second panel and may be between approximately 0 degrees and approximately 180 degrees. The angled intermediate position may allow insertion of the needle and/or catheter without the angle of insertion having to be manually maintained.

The construction material of the guidance device 100, particularly for the first panel 110 and the second panel 120, may be chosen based on a desired balance of flexibility and rigidity and may be or may include a type of plastic, rubber, and/or the like. The chosen material may allow the panels to conform subtly to the contours of a patient's skin, thereby enhancing comfort and fit, while simultaneously retaining sufficient rigidity to facilitate accurate alignment and secure positioning of a medical apparatus, such as needle 210 or catheter 220. Alternative materials may also be considered, provided they meet the conformability to skin contours and maintenance of structural integrity for device alignment.

In some embodiments, first panel 110 includes opposing top and bottom surfaces. The first panel 110 may include an attachment element 112, which may serve to secure the first panel 110, and in turn the guidance device 100, to a first surface, such as the skin of a patient. The attachment element 112 may be located along one or more surface of the first panel, and the attachment element 112 may occupy a portion or the entirety of the one or more surface of the first panel. FIG. 1 depicts the attachment element 112 as attached and/or integrated with the bottom surface of the first panel 110.

In some embodiments, the attachment element 112 may be an adhesive element. The attachment element 112 may be a dual-sided adhesive, where a first side of the dual-sided adhesive is adhered to the bottom surface of panel 110, while a second side of the dual-sided adhesive is configured to be adhered to an external surface, such as the skin of a patient. The attachment element 112 may include a thin, low-adhesive structure which covers the second side of the dual-sided adhesive prior to application to an external surface. In some embodiments, the placement of the guidance device 100 includes identifying a target of entry associated with a patient, aligning the target region 140 with the target of entry associated with the patient, and utilizing the attachment element 112 to attach the first panel 110 to skin of the patient, such that the target region 140 aligns with the target of entry associated with the patient.

In some embodiments, the first panel 110 includes one or more locking elements 114, which, in some embodiments, may be referred to as first or second engagement elements. These locking elements 114 are typically situated on a surface of the first panel 110 that opposes the attachment element 112, such as the top surface of the first panel 110, as illustrated in FIG. 1. The locking elements 114 can be located at various positions along the surface of the first panel 110. The design of each locking element 114 may incorporate a protrusion or extension that is configured to engage with the receiving elements 124 located on the second panel 120, which, in some embodiments, may be referred to as first or second engagement elements. When the guidance device 100 transitions from an unlocked configuration to a locked configuration, the locking elements 114 interlock with the receiving elements 124, thereby inhibiting relative movement between the first panel 110 and the second panel 120. It will be appreciated that the relative positioning of the locking elements 114 and the receiving element 124 are shown as being associated with the first and second panels, respectively, but that the guidance device 100 is operable with locking elements 114 positioned on the second panel 120 and the receiving elements 124 positioned on the first panel.

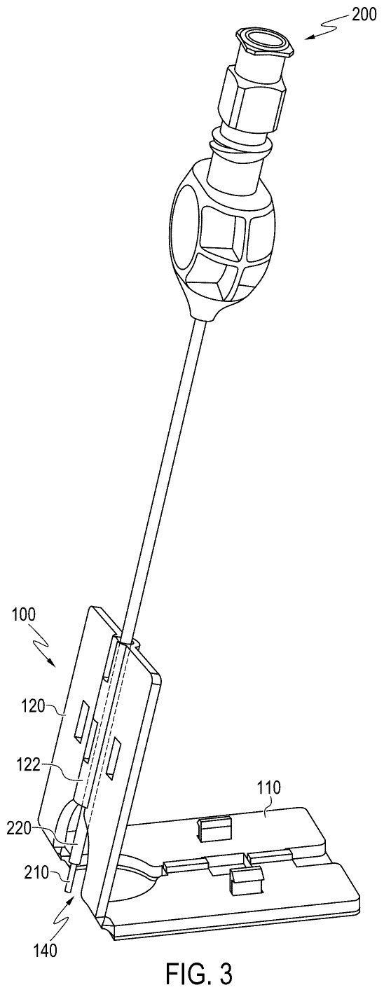

In some embodiments, the first panel 110 may include one or more friction member 116, which may be positioned along the midline of panel 110. One or more friction member 116 may be integrally formed with the first panel 110 or separately attached, and a portion of it may protrude above the top surface of the first panel 110. The construction of the friction member 116 can vary; it may be made from the same material as the first panel 110, ensuring uniformity, or from a different material, selected to enhance specific characteristics like durability or frictional properties. The material of the friction member 116 may be chosen for its rigidity or its capacity to be resiliently deformable. In scenarios where it is resiliently deformable, the friction member 116 is designed to deform under a compression force, where the one or more friction member 116 exerts a reactive force that provides a friction coupling between the one or more friction member 116 and the outside object which is applying the compression force. Furthermore, the friction member 116 is designed to align with the cutouts 126 found in the guidance element 122 of the second panel 120. This alignment plays a role in the functioning of the guidance device 100, especially when it is in the locked configuration. When a medical device 200, such as needle 210 or catheter 220, is interfaced with the guidance element 122, the friction member 116 interacts with the cutouts 126 to effectively capture and restrict the movement of the medical device 200. This interaction ensures that the medical device, such as catheter 220 (e.g., as shown in FIG. 3), is securely held in place, reducing the likelihood of displacement during and after a medical procedure.

The first panel 110 may further include one or more voids 117. Voids 117 may manifest as cutouts that either extend entirely through a depth of the panel or only partially. These voids 117 are, in some embodiments, positioned along the midline of the panel, adjacent to or interspersed among one or more friction members 116. Their placement may align with one or more protrusions that might be associated with the guidance element 122 of the second panel 120 when the guidance device 100 is in the locked configuration. This alignment allows for the interaction between the first panel 110 and the second panel 120, especially when guiding and securing a medical device such as needle 210 or catheter 220 (as shown in FIG. 3) in place, and further allows a secure friction interaction between friction members 116 and a medical device when in the locked position.

In some embodiments, the second panel 120 may include a guidance element 122. This guidance element 122 may be positioned along the midline of the second panel 120. Its primary function is to receive one or more medical devices, as exemplified in FIG. 3. The design and positioning of the guidance element 122 are such that it can accommodate medical devices like needles or catheters, facilitating their insertion and positioning during medical procedures. The guidance element 122 may be shaped or configured in various ways to ensure compatibility with different types and sizes of medical devices.

The guidance element 122, as part of the second panel 120, may be generally cylindrical in shape. This cylindrical structure may extend beyond one or both the top and bottom surfaces of the second panel 120, as depicted in FIGS. 1 and 2. Incorporated into the walls of this cylinder are one or more cutouts 126. These cutouts 126 are designed to facilitate interaction with the friction members 116 and voids 117 of the first panel 110. This interaction allows the guidance element 122 to effectively interlock with the friction members 116 and align with the voids 117, contributing to the stable positioning and orientation of the guidance device 100 when in use. The cylindrical shape of the guidance element 122, combined with the strategically placed cutouts 126, provides a structured pathway for guiding medical devices, while allowing for secure engagement with the corresponding features of the first panel 110.

As previously discussed, the second panel 120 may also include one or more receiving elements 124, which have been previously mentioned in the context of their interaction with the locking elements 114 of the first panel 110. These receiving elements 124 are designed to engage with the locking elements 114 when the guidance device 100 transitions into its locked configuration. This engagement ensures that the first panel 110 and the second panel 120 are secured relative to each other, preventing unintentional movement. Generally, receiving elements 124 may be cutouts in the second panel 120 which receive one or more of the locking elements 114, which may be in form of a clip as depicted in FIG. 1.

In some embodiments, hinges 130 serve as the connecting mechanism between the first panel 110 and the second panel 120 in the guidance device 100. As illustrated in FIG. 1, there may be two hinges, which may be positioned on opposing sides of the target region 140. These hinges 130 allow for the relative movement of the first panel 110 and second panel 120, allowing them to be adjusted as needed for the specific requirements of a medical procedure. The placement of the hinges 130 is such that they facilitate the opening and closing motion of the panels, akin to a book, while maintaining the alignment of the target region 140. This configuration allows for the flexible positioning of the first panel 110 and second panel 120 while ensuring they remain interconnected throughout the operation of the guidance device 100. Although two hinges 130 are shown in FIGS. 1 and 2 and generally discussed herein, it will be understood that any applicable number (e.g., one or more) of hinges 130 may be used to implement the systems and methods disclosed herein.

As discussed herein, guidance device 100 may be configured in an angled intermediate position. Such an angled intermediate position may be held (e.g., temporary locked) based on cut outs (not shown) or force components associated with or in contact with hinges 130. Such components may, for example, apply a counterforce against a force directing second panel 120 to move towards and/or away from the first panel 110. Such a counterforce may maintain an angle of the second panel 120 relative to the first panel 110 during insertion of a catheter and/or needle such that the angle of insertion of the catheter and/or needle is maintained at the angled intermediate position.

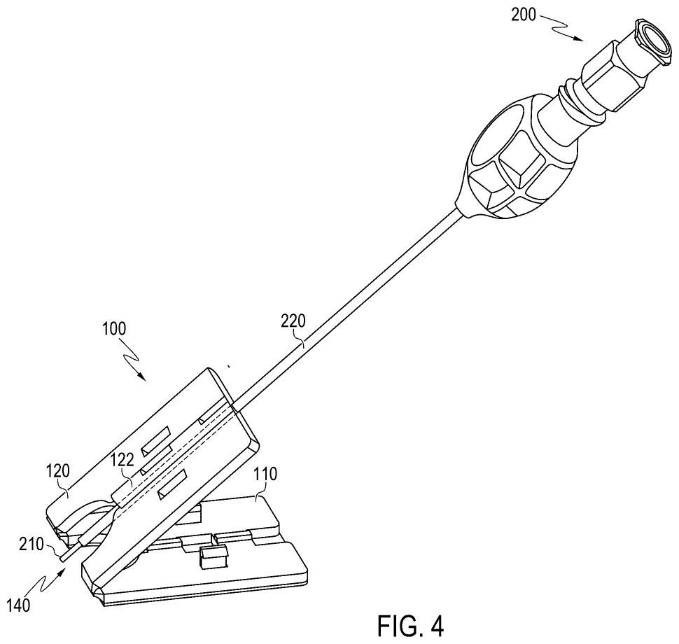

In some embodiments, the first panel 110 and second panel 120 may each include a cutout which at least partially defines target region 140. These cutouts, when aligned, collectively form the target region 140, which may be the focal point for the medical device when positioned in the guidance element 122. The design of these cutouts ensures that as the second panel 120 rotates about the hinges 130, the orientation of the medical device can be adjusted while its trajectory remains consistently directed towards the central area of the target region 140, such as can be seen in FIGS. 3 and 4. This setup is particularly useful for procedures where the angle of entry of a medical device, such as a needle or catheter, needs to be varied, while still precisely targeting a specific entry point on the patient. The configuration of the target region 140, in conjunction with the adjustable panels, facilitates accurate alignment and positioning of the medical device relative to the desired entry point.

Referring to FIGS. 3-7, the use of the guidance device 100 in conjunction with the medical device 200 is shown. In some embodiments, a user may identify a desired point of entry on a patient for the medical procedure. Once this point is determined, the adhesive (e.g., attachment element 112) on the first panel 110 is prepared for use, which may involve actions such as removing a protective covering from the adhesive element. Following this preparation, the first panel 110 is then adhered to the patient's skin. During this process, care is taken to align the center of the target region 140 with the identified point of entry.

In some embodiments, following the securement of the first panel to the patient, the step involves the insertion of the medical device 200, which, as depicted in FIG. 3, includes components such as the needle 210 and/or the catheter 220. This insertion is performed through an opening in guidance element 122 located in the second panel 120. The medical device 200 is introduced into the guidance element 122 from an end of the second panel 120 that is opposite to the hinges 130. This orientation results in the medical device 200 being aligned towards the desired point of entry on the patient. The design of the guidance element 122 aids in guiding the needle 210 and catheter 220, facilitating precise alignment with the target region 140, which corresponds to the intended entry point on the patient's body.

In some embodiments, as illustrated in FIG. 4, the user has the option to manipulate the medical device 200 to achieve a specific insertion angle. This manipulation involves rotating the second panel 120 in relation to the first panel 110. The flexibility provided by the hinges 130 allows for this adjustment of the second panel 120, allowing the user to set the medical device at various angles (e.g., an angled intermediate position) while targeting the desired point of entry associated with the target region 140. Due to the first panel 110 being adhered to the patient's skin and the medical device 200 being inserted into the guidance element 122, the orientation of the medical device consistently points towards the predetermined entry point, regardless of the angle of insertion. This consistent targeting facilitates ease of use for the practitioner, allowing them to find the desired angle of insertion with one hand, while freeing up the other hand for additional tasks. For example, the user may simultaneously operate an ultrasound device to assist in determining the most suitable angle of insertion. This capability enhances the practicality and efficiency of the procedure, providing the user with greater control and flexibility during the medical process.

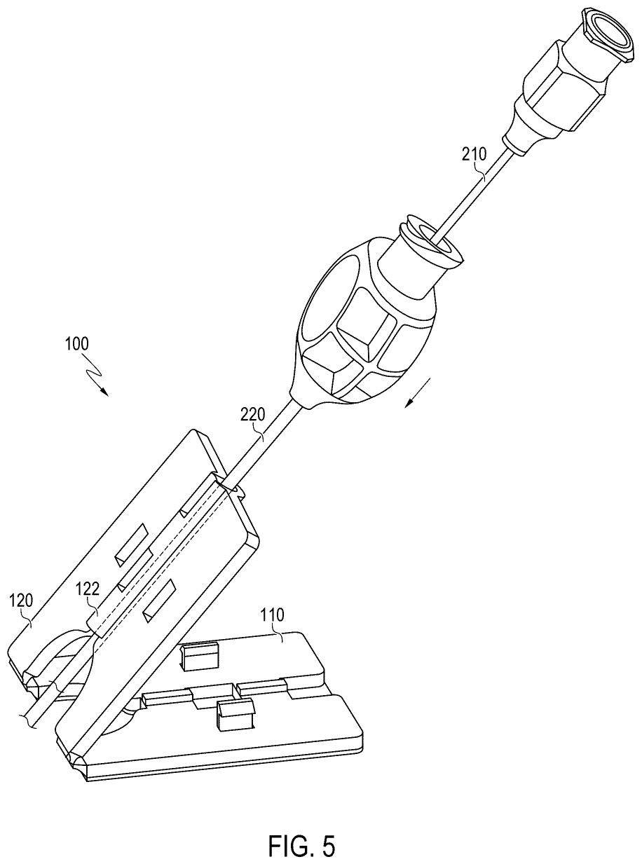

In some embodiments, as illustrated in FIG. 5, the method may then include the user inserting at least a portion of the medical device 200 into the patient at the desired angle. This insertion involves moving the medical device 200 in a first direction towards the target point of entry. The guidance device 100, particularly due to its stable positioning and alignment capabilities, assists the user in maintaining both the targeted entry point and the chosen angle of insertion. As a result, this step can be executed with one hand, offering the user the convenience of maintaining the desired angle and position without needing to manually hold or adjust the guidance device 100 and/or the medical device 200. In this step, both the needle 210 and catheter 220 may be inserted at the target point of entry, either together or with the insertion of the needle 210 first, and the insertion of the catheter 220 second.

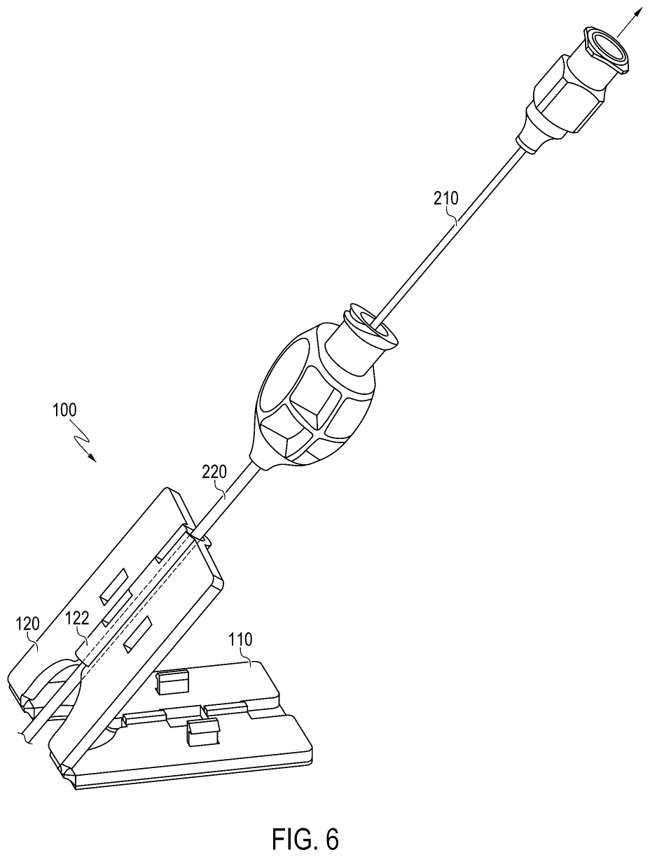

In some embodiments, as illustrated in FIG. 6, the method may then include the user withdrawing a portion of the medical device 200, such as the needle 210, in a second direction. This action is distinct from the initial insertion movement and involves pulling the needle 210 back while ensuring that the catheter 220 remains inserted at the desired location within the patient. The withdrawal of the needle 210 is executed in a manner that carefully removes it both from the patient and from the guidance element 122 of the guidance device 100. The design and functionality of the guidance device 100 support this movement by maintaining the position of the catheter 220, even as the needle 210 is being withdrawn.

Although FIGS. 3-5 provide an example of using guidance device 100 with the medical device 200 where the medical device 200 includes the catheter 220 through which the needle 210 is inserted (e.g., a catheter-over-needle assembly), it will be understood that the guidance device 100 may be used with different medical devices. For example, the guidance device 100 may allow insertion of a catheter-through-needle assembly using the same or similar methods and components discussed herein.

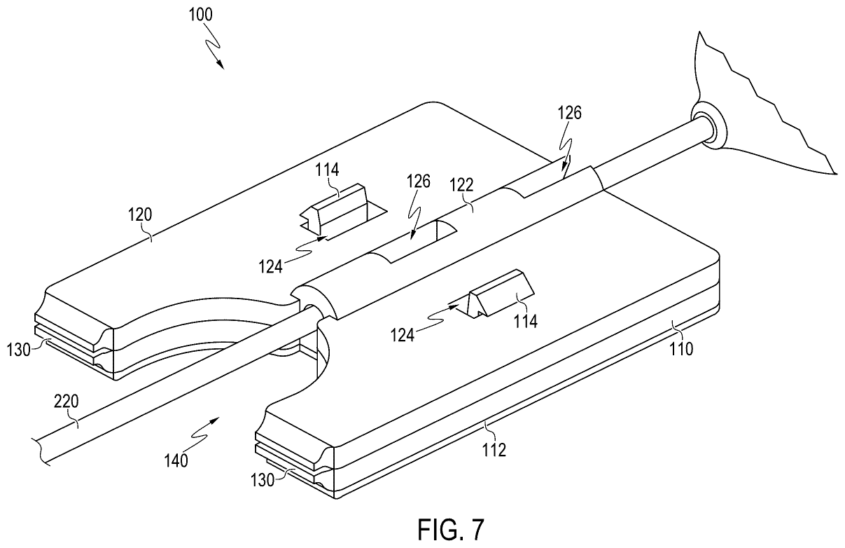

In some embodiments, the method may then include moving the guidance device 100 from an unlocked configuration into a locked configuration, as shown in FIG. 7. This transition involves rotating the second panel 120 towards the first panel 110, facilitating the coupling of the locking elements 114 with the receiving elements 124. As this rotation occurs, the guidance element 122, particularly its cutouts 126, becomes aligned with the friction members 116 of the first panel 110. With the catheter 220 still positioned within the guidance element 122 during this process, the alignment of these cutouts 126 with the friction members 116 results in the catheter being effectively restricted in movement. This restriction stabilizes the catheter 220 and securely holds the catheter 220 in place while the first panel 110 remaining adhered to the skin of the patient. As discussed herein, this entire process may be completed by a user with a single hand, freeing them to complete other tasks or utilizes additional equipment during the placement of the catheter 220. Alternatively, according to an embodiment, one or more of the steps discussed herein may be conducted using an automated system such as, for example, a robotic arm and related components. According to some embodiments, guidance device 100 may transition from a locked position to an unlocked position by uncoupling locking elements 114 from receiving elements 124. Locking elements 114 may be sized and/or shaped to allow a user to uncouple locking elements 114 from receiving elements 124. For example, locking elements 114 may include a component that extends at least approximately half an inch from a surface of the first panel 110 and shaped to allow a user's fingers to uncouple locking elements 114 from receiving elements 124 using a single hand. A user may, for example, uncouple locking elements 114 from receiving elements 124 to reposition or remove catheter 220.

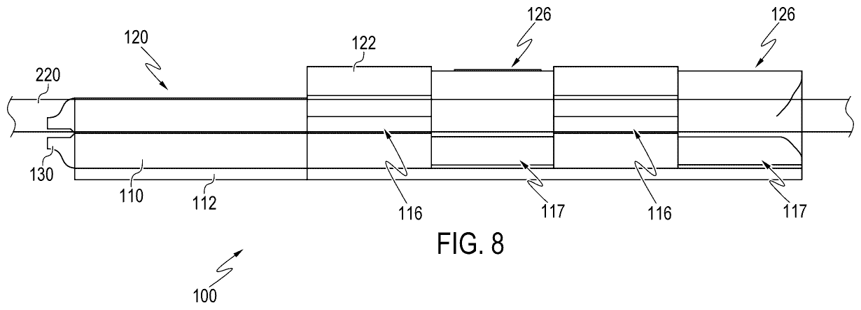

Referring to FIG. 8, a cross-section view of the guidance device 100 with catheter 220 is provided. In FIG. 8, catheter 220 is shown as secured by the one or more friction members 116. The guidance element 122, housed within the second panel 120, may be seen aligning its cutouts 126 with the friction members 116 of the first panel 110. This alignment indicates that the catheter 220, which passes through the guidance element 122, is in direct contact with, and is being restrained by, the friction members 116 and one or more portion of the guidance element 122, such as an interior surface and/or interior wall of the guidance element 122. The friction members 116, which may be resiliently deformable, apply a stabilizing force onto the catheter 220, thereby reducing its potential for undesired movement. Additionally, the cross-sectional view in FIG. 8 demonstrates, in some embodiments, the relative position of the voids 117 in the first panel 110, which are adjacent to or in between the friction members 116. These voids 117, while not directly engaging with the catheter 220, contribute to the overall structure and functionality of the guidance device 100 in its locked state by receiving portions of the guidance element 122.

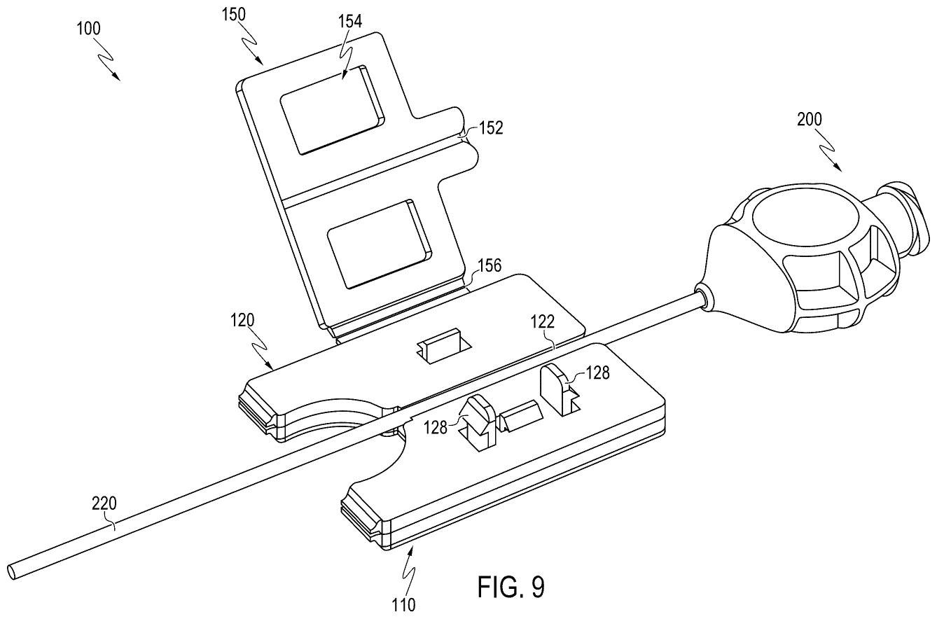

Referring to FIGS. 9-12, in some embodiments, the guidance device 100 is configured to include a third panel 150, which may function as a securing panel. This third panel 150 may operate in conjunction with the second panel 120 to selectively secure aspects of a medical device 200, such as a needle or catheter, within a guidance pathway.

In such a configuration, in some embodiments, the second panel 120 may incorporate one or more locking elements 128. These locking elements 128 may protrude in a direction opposing the first panel 110, thereby facilitating the engagement with the third panel 150. The locking elements 128 may be configured, as depicted in FIG. 9, as hooks, which can include, but are not limited to, snap-fit hooks. The structural design of these hooks is such that they are capable of being received by and interlocking with receiving elements 154 located on the third panel 150. This interlocking mechanism contributes to the stability and security of the medical device 200 when positioned within the guidance pathway. The snap-fit nature of the locking elements 128 provides a secure yet detachable connection, allowing for easy assembly and disassembly as required. Embodiments disclosed herein are not limited to the snap-fit hooks shown in FIG. 9. It will be appreciated that alternative, selectively coupling connections are contemplated and may be utilized, depending on the specific nature and configuration of the guidance device 100.

In some embodiments, such as those depicted in FIGS. 9-12, guidance element 122 includes an open side. The general structure and function of the guidance element 122 in these embodiments remain consistent with those described in previous configurations, with the variation the open side, which is depicted in FIGS. 9-12 as being a side of the second panel 120 opposite the side which abuts the first panel 110 in the closed configuration. The guidance element 122, characterized by its open side, aligns with the guidance element 152 when the third panel 150 is rotated to a position flush with the second panel 120. This alignment creates a continuous, unified pathway through which a medical device, such as catheter 220, can be inserted, swapped, or otherwise manipulated. The structural integration of these two elements ensures that the pathway remains stable and precise in its guidance function, akin to the pathways of previous embodiments. This open side design of the guidance element 122 allows for an enhanced level of accessibility and allows removal of the medical device 200 from the guidance element 122, swapping in a new medical apparatus, securing the medical apparatus by closing the guidance pathway by securing the third panel 150 to the second panel 120, and then by operating the guidance device 100 in any one or more of the manners described herein.

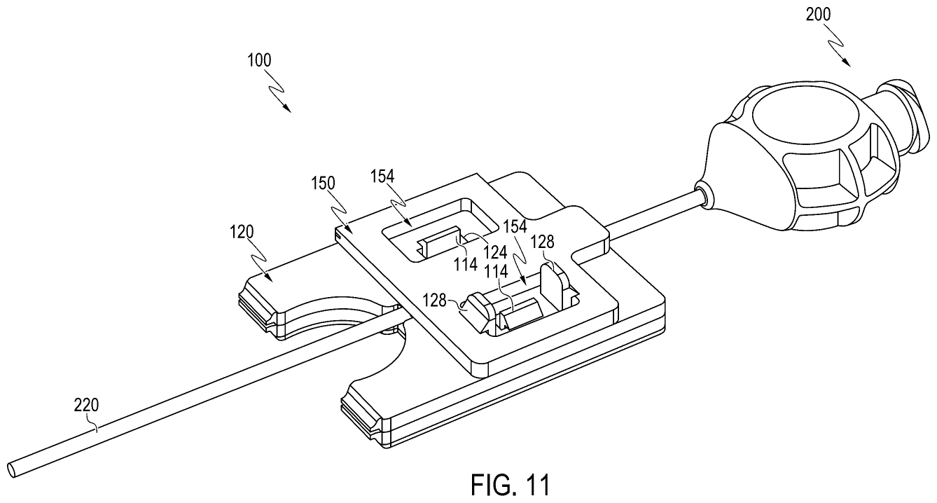

In some embodiments, utilizing the guidance device 100, as detailed in FIGS. 9-12, the initial placement of a catheter into a patient and the subsequent securing of the guidance device 100, inclusive of the catheter 220, follows a procedure similar to that described in prior embodiments. This initial step involves aligning the guidance device 100 with the target entry point on the patient's skin and securing the first panel 110, usually through an attachment element 112, to ensure stable positioning. The resulting configuration, when utilizing the embodiment of FIGS. 9-12, results in the third panel 150 being in a closed configuration and the guidance device 100 being in a locked configuration, such as depicted in FIG. 11.

Further to the initial placement of the catheter 220, the method may include the operation of moving the third panel 150 from a closed to an open position. This action is facilitated by selectively disengaging the third panel 150 from the second panel 120. The disengagement process involves manipulating the locking elements 128 on the second panel 120 and the corresponding receiving elements 154 on the third panel 150. These elements, which may be designed as snap-fit components or similar configurations, allow for a secure yet reversible connection. The movement of the third panel 150 to an open position, which may occur by further rotating the third panel relative to the second panel around one or more hinges 156, effectively exposes the guidance element 122 on the second panel 120, which is designed with an open side in this embodiment. This exposure, and the third panel being in the open position, is shown in FIG. 9.

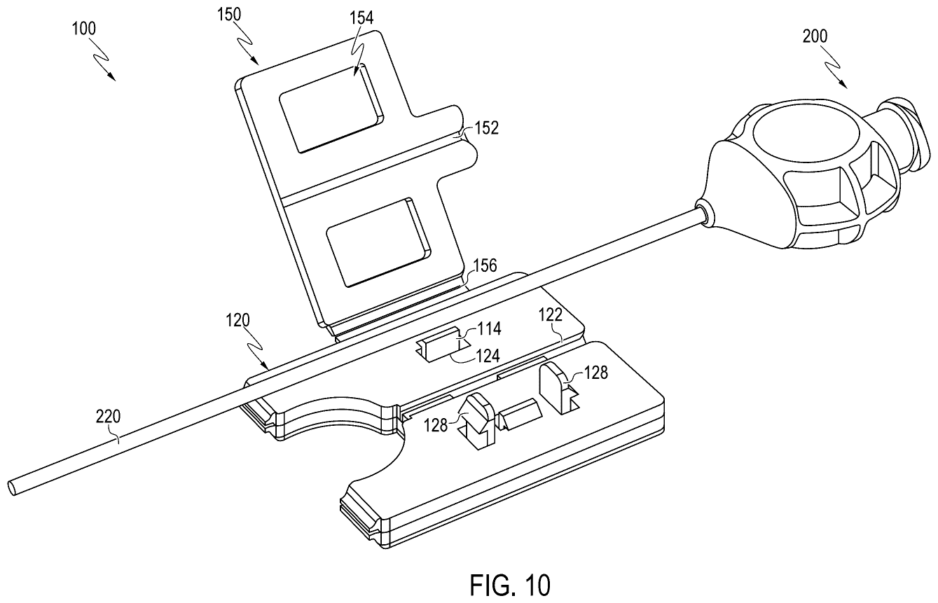

In some embodiments, as depicted in FIG. 10, the guidance device 100 allows the catheter 220 to be lifted out of or otherwise removed from the guidance element 122. The lifting of the catheter 220 is enabled by the open design of the guidance element 122 on the second panel 120, which becomes accessible once the third panel 150 is moved to an open position. This capability to lift or otherwise remove the catheter 220 out of the guidance element 122 presents significant advantages in medical procedures. It permits the repositioning or swapping of the catheter 220 for one or more other medical devices. For instance, a practitioner may choose to reposition or replace an existing catheter 220 with a new catheter placement assembly (e.g., a medical device 200), which may include a new needle 210 (as shown in FIGS. 3-5) and a second catheter (not shown). Such interchangeability is particularly beneficial where the replacement of the catheter becomes necessary due to procedural adjustments or the specific medical needs of the patient. The design of the guidance device 100, in this embodiment, facilitates this swapping process without necessitating the removal of the entire device from the patient's skin, thereby maintaining the original placement and alignment.

In some embodiments, as depicted in FIG. 11, the guidance device 100 illustrates a specific arrangement of securing elements 128 and receiving elements 154. Two securing elements 128 are shown, positioned on opposing sides of the second panel 120. These securing elements 128 are configured to interact with corresponding receiving elements 154 located on the third panel 150. The receiving elements 154 on the third panel 150 are designed as voids or cutouts, placed to align with the receiving elements 124 of the second panel 120 and to engage with the securing elements 128.

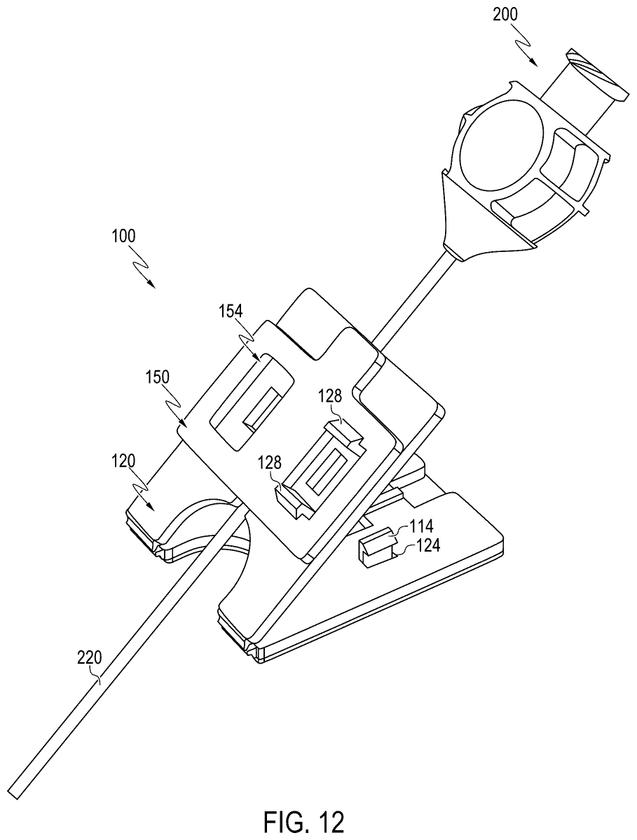

This alignment between the receiving elements 154 of the third panel 150 and the receiving elements 124 of the second panel 120 serves a dual purpose. First, it ensures a secure connection between the second and third panels when the third panel 150 is in a closed position, thereby stabilizing the guidance pathway for the medical device 200. Second, this alignment facilitates user access to the locking elements 114 of the first panel 110. When the third panel 150 is in a closed configuration, the user can easily reach and manipulate the locking elements 114 (e.g., using one hand). This accessibility allows disengaging the first panel 110 from the second panel 120, a step that may be desired during certain stages of a medical procedure or when adjusting the position or configuration of the guidance device 100. For example, a method may include disengaging the second panel from the first panel and moving the guidance device 100 to an unlocked configuration for placement or insertion of a fresh catheter 220 into the patient. This may involve the need to further align an entry angle, as previous described. The ability to disengage the first and second panel after the insertion of the new medical device 200 and moving the third panel into the closed position allows this additional alignment, which is depicted in FIG. 12, without deviating from the desired target of entry. After the alignment is moved to a desired angle, the method proceeds with insertion and moving from an unlocked to a locked position, as previously described.

While the third panel 150 is shown as hingedly connected to the second panel 120, in some embodiments, the third panel 150 of the guidance device 100 may be configured to slide between an open and closed configuration. This sliding mechanism facilitates the transition of the third panel 150 in relation to the second panel 120, allowing for access to the guidance element 122. The design of the sliding mechanism may include rails, tracks, or similar guiding structures that ensure smooth, controlled movement of the third panel 150. The sliding configuration is configured to maintain alignment of the receiving elements 154 with the securing elements 128, ensuring that when the third panel 150 is in the closed position, it securely locks in place, thereby stabilizing the guidance pathway formed in conjunction with the guidance element 122 of the second panel 120.

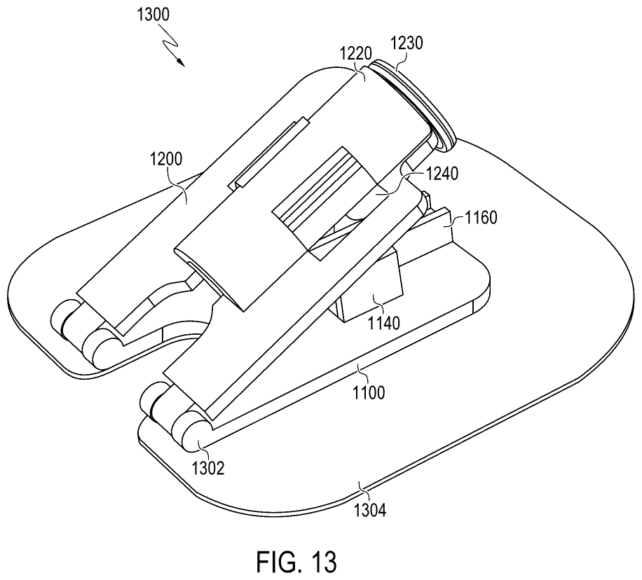

In some embodiments, an alternative configuration of the guidance device discussed herein is provided, as illustrated in FIGS. 13-18J. This embodiment shares some features and principles with the previously described guidance devices, while incorporating distinct elements and methodologies. A guidance device 1300, as shown in FIG. 13 includes a first panel 1100, a second panel 1200, and one or more hinges 1302 that connect the first and second panels.

First panel 1100 of guidance device 1300 includes one or more locking elements 1140, which are generally similar in function to locking elements 114 described in previous embodiments. Additionally, first panel 1100 incorporates rails 1160 that extend longitudinally along an axis of the first panel. Together, these rails 1160 form a path or receiving area along an axis (e.g., a centerline) of first panel 1100, which is configured to receive and retain/restrict one or more elements of a medical apparatus, such as a needle, guide wire, or catheter.

Second panel 1200 of the guidance device 1300 features a guidance element 1220, which is formed into the second panel such that it may be constructed as an arc or other shape that defines an internal pathway. This internal pathway is designed to accommodate additional elements, such as a collar 1230. The collar 1230 is a device (e.g., a cylindrical shaped device) configured to be received by the guidance element 1220, serving to aid in the guidance and placement of a medical apparatus. By reducing the size of the internal pathway of the guidance element 1220, the collar 1230 facilitates more precise placement. The disclosure considers different sized collars, allowing the same guidance element 1220 and guidance device 1300 to be used with medical apparatus of various sizes by changing the inner diameter of the collar 1230.

Hinges 1302 may be rotatable in multiple dimensions such that, for example, second panel 1200 is rotatable in a traverse direction relative to first panel 1100. Accordingly, hinges 1302 may allow first panel 1100 and second panel 1200 to move rotationally (e.g., between an open and closed configuration) and may further allow rotation of first panel 1100 and second panel 1200 in a traverse (e.g., left to right or right to left) direction. Hinges 1302 may be lockable in the rotational direction, as discussed herein and may further be lockable in the traverse direction.

Second panel 1200 also includes receiving elements 1240, which are similar to the receiving elements 124 described earlier. These receiving elements 1240 are configured as voids positioned through the surface of the second panel 1200 and may be located on opposing sides of the guidance element 1220. In some embodiments, the receiving elements 1240 may be partially incorporated within the guidance element 1220, such that the barrel of the guidance element includes a ridge or recess to accommodate a portion of the locking elements 1140, thereby securing the locking elements to the receiving elements through means such as a friction fit.

Hinges 1302 in this embodiment of the guidance device 1300 are formed into the first panel 1100 and second panel 1200, but not as living hinges. Instead, the first and second panels are separate pieces that are joined together after manufacturing. The perimeters of the first and second panels may align when the guidance device 1300 is in a locked configuration. In some embodiments, a portion of the second panel 1200 extends beyond the perimeter of the first panel 1100 in the locked configuration, creating a space between the surface (e.g., the skin of the patient) and the second panel. This space allows a practitioner to gain leverage to unlock the first and second panels when the locking mechanism is a friction fit. Alternatively, in some embodiments, a portion of the first panel 1100 may extend beyond the perimeter of the second panel 1200 in the locked configuration, creating two tabs. These tabs allow a practitioner to apply forces in opposite directions on the first and second panels, respectively, to disengage the locking elements from the receiving elements.

The rails 1160 of the first panel 1100 are configured to extend longitudinally along the axis of the first panel 1100, typically positioned parallel to each other and equidistant from the centerline of the panel. The rails 1160 may be integrally formed with the first panel 1100 or separately attached, depending on the manufacturing process and materials used. The height of the rails 1160 may vary but generally protrude above the surface of the first panel 1100 to a degree sufficient to create a path or receiving area between them.

The path or receiving area formed by the rails 1160 is designed to receive and retain or restrict one or more elements of a medical apparatus, such as a needle, guide wire, or catheter. When such a medical apparatus is placed between the rails 1160, the rails serve to limit lateral movement of the apparatus, ensuring it remains in a consistent, predetermined position relative to the centerline of the first panel 1100. This restriction of lateral movement is particularly beneficial when the guidance device 1300 is in a locked configuration, as it aids in maintaining the stability and precise positioning of the medical apparatus.

The spacing between the rails 1160 may be customized to accommodate different sizes of medical apparatus. In some embodiments, the guidance device 1300 may be produced with rails 1160 at a standard spacing suitable for the most commonly used sizes of needles, guide wires, or catheters. In other embodiments, the rails 1160 may be adjustable, allowing the practitioner to adapt the guidance device 1300 to different medical apparatus as needed. This adjustability could be achieved through various mechanisms, such as sliding the rails 1160 along tracks or swapping out interchangeable first panels 1100 with rails 1160 at different spacing.

The guidance element 1220 is formed into the second panel 1200 and shaped as an arc or cylinder that defines an internal pathway. This pathway is designed to guide and position a medical apparatus, such as a needle, guide wire, or catheter, during a medical procedure. The arc shape of the guidance element 1220 allows for a smooth, controlled transition of the medical apparatus from the second panel 1200 to the first panel 1100 and to the targeted entry point on the patient's skin.

The internal pathway of the guidance element 1220 is sized to accommodate the medical apparatus being used. In some embodiments, to enhance the versatility of the guidance device 1300, the system includes an additional component: the collar 1230. The collar 1230 is a cylindrical device designed to be received within the internal pathway of the guidance element 1220. It functions to adapt the size of the internal pathway, thereby allowing the use of an initial medical device, such as a needle, that may be of a different size than the catheter or sheath ultimately or subsequently secured at the end of the placement procedure. For example, the collar 1230 may be used to accommodate a smaller gauge needle during the initial insertion, while subsequently allowing for the placement of a larger diameter catheter or sheath over the guide wire.

The collar 1230 can be inserted into the guidance element 1220 prior to the introduction of the medical apparatus. Once in place, the collar 1230 creates a smaller, more restrictive pathway within the guidance element 1220. This reduced pathway size is beneficial when using smaller gauge needles, guide wires, or catheters, as it helps to prevent unwanted lateral movement or deflection of the apparatus during insertion.

The use of the collar 1230 allows a single guidance element 1220 to be adapted to various sizes of medical apparatus. Rather than requiring multiple guidance devices 1300 with different sized guidance elements 1220, the practitioner can simply select the appropriate collar 1230 for the apparatus being used. This adaptability streamlines the procedure and reduces the need for additional equipment.

The collar 1230 may be made from various materials, depending on the specific requirements of the procedure and the medical apparatus being used. In some embodiments, the collar 1230 may be constructed from a rigid, durable material, such as a hard plastic or metal, to provide maximum stability and precision. In other embodiments, the collar 1230 may be made from a softer, more flexible material, such as silicone or a soft plastic, to allow for a more secure, custom fit around the medical apparatus. The collar 1230 may be designed as a single-use, disposable component or as a reusable component that may be sterilized.

In some embodiments, the collar 1230 may include additional features to enhance its functionality or ease of use. For example, the collar 1230 may have a flared or flanged end to facilitate insertion into the guidance element 1220, or it may include gripping surfaces or tabs to aid in removal from the guidance element 1220.

The method of using the guidance device 1300 may begin with securing the first panel 1100 to a surface 1304, such as the skin of a patient, as depicted in FIG. 13. This step involves aligning the target area, typically defined by a cutout or marking on the panel, with the desired point of entry on the patient's skin. Securing the first panel 1100 may involve the use of an adhesive, straps, or other fastening means as discussed herein, depending on the specific design of the guidance device 1300.

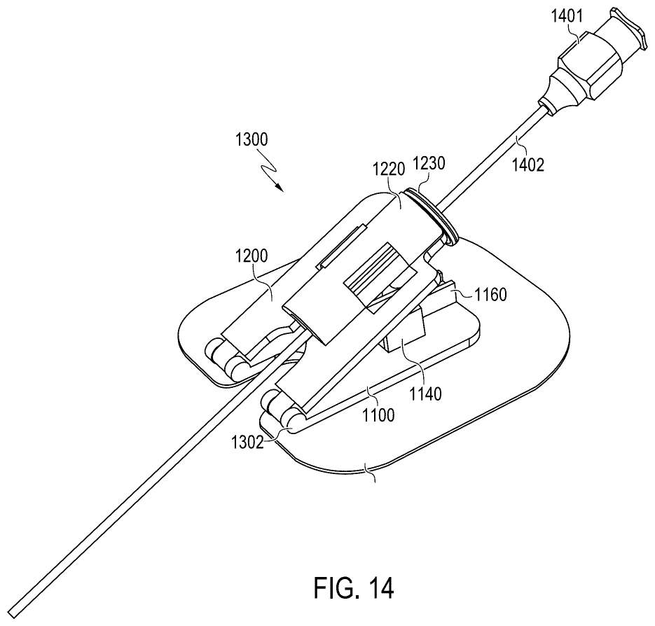

Once the first panel 1100 is in place, the next step involves inserting a component 1402 of a medical apparatus 1401 into the guidance element 1220 of the second panel 1200, as shown in FIG. 14. The component 1402 is also passed through the collar 1230, which is positioned within the guidance element 1220. The component 1402 may include one or more of a needle, catheter, or guide wire. The angle of insertion may be adjusted by manipulating the second panel 1200 relative to the first panel 1100, using the hinges 1302 as a pivot point.

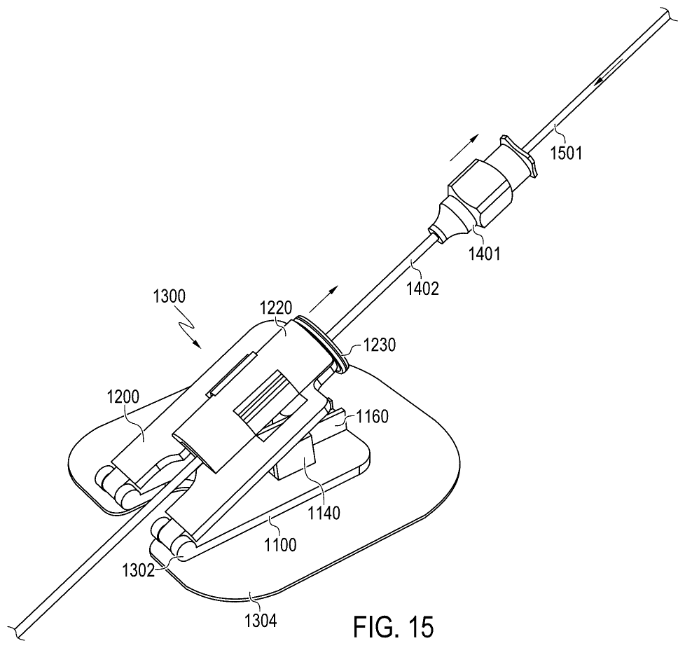

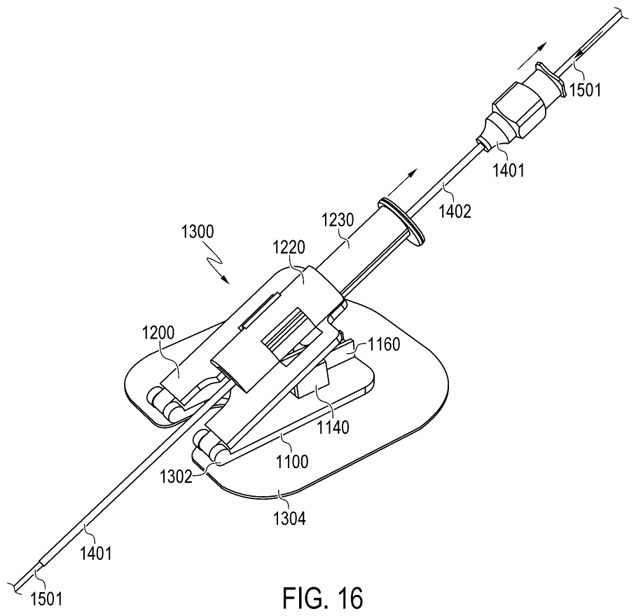

FIGS. 15 and 16 illustrate a scenario in which a guide wire 1501 is inserted via component 1402. After component 1402 is inserted into the patient's skin at the target entry point, guide wire 1501 is then inserted through component 1402. Once guide wire 1501 is in place, the medical apparatus 1401 including component 1402 is withdrawn from the guidance device 1300, leaving the guide wire 1501 inserted in the patient and threaded through the guidance element 1220.

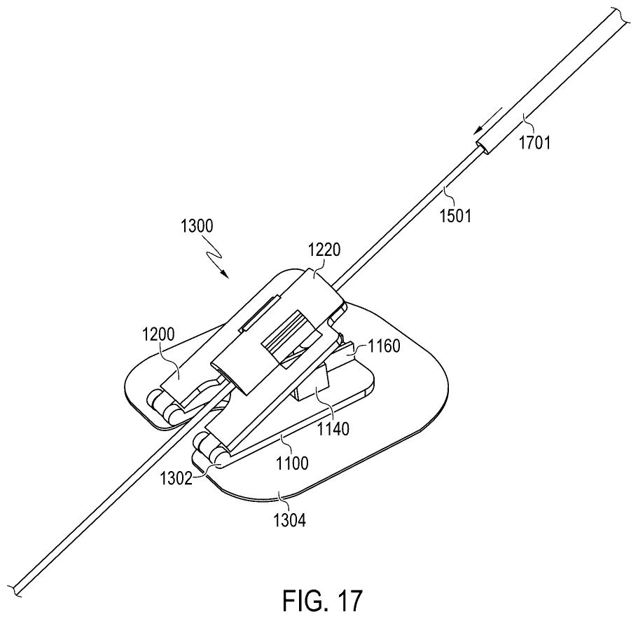

FIG. 17 demonstrates inserting a sheath 1701 (e.g., a dialing sheath). Sheath 1701 may be thicker than component 1402. Sheath 1701 is slid over guide wire 1501, passing through the guidance element 1220 and into the patient. Once the sheath 1701 is in place, guide wire 1501 is removed. This multi-step process allows the placement of component 1402 and guide wire 1501, followed by the placement of a sheath 1701 of a desired size. Guide wire 1501 serves as a pathway for the sheath 1701, and the practitioner can change the size of the catheter as needed without repeating the entire insertion process.

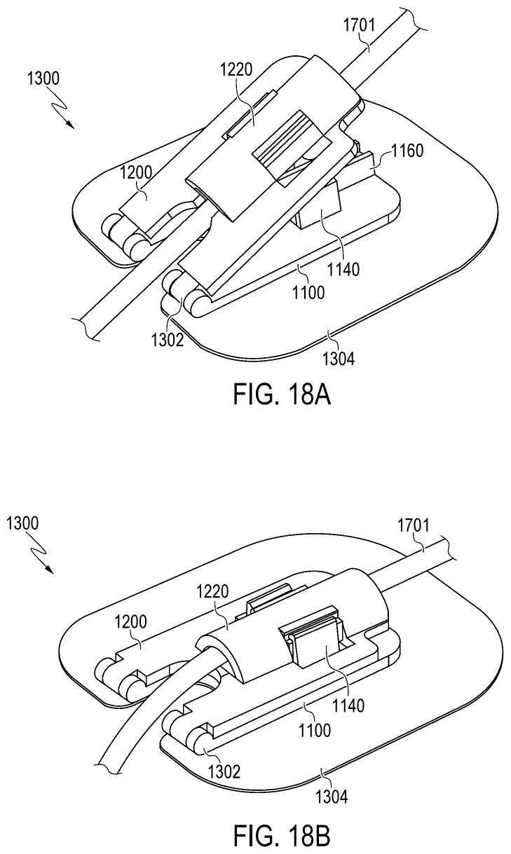

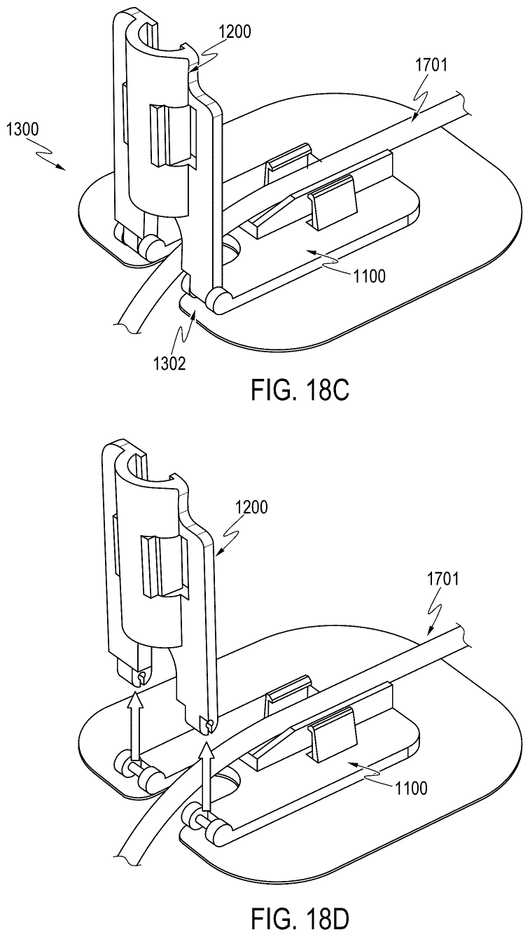

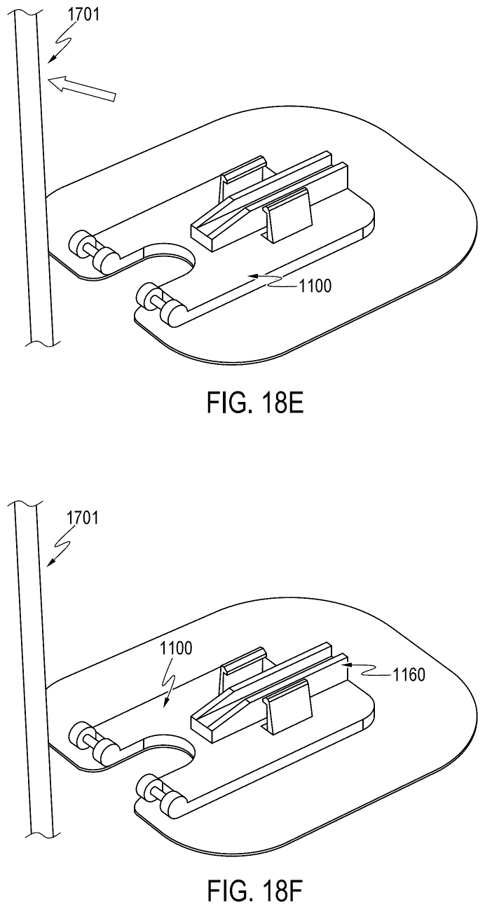

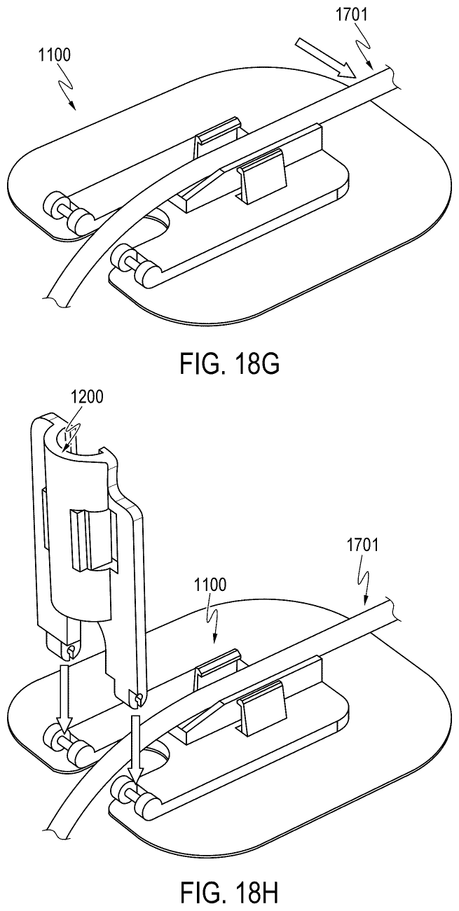

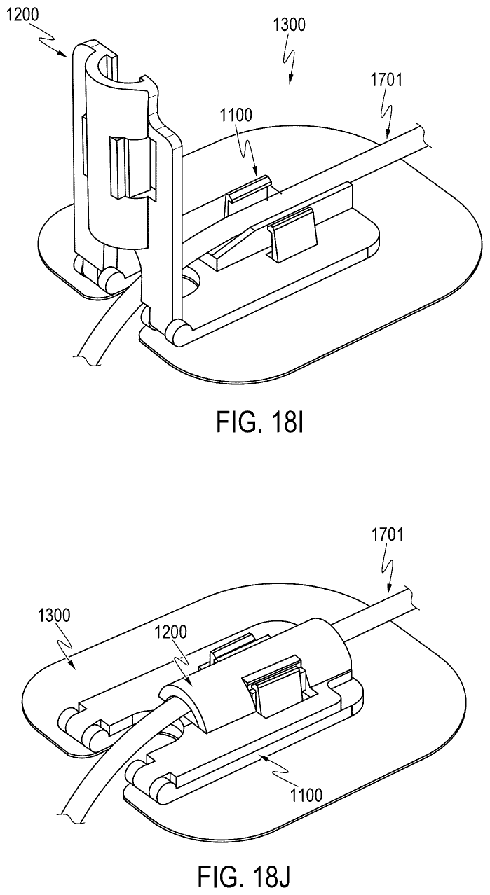

FIG. 18A shows the guidance device 1300 in an intermediate unlocked state, and FIG. 18B shows the guidance device 1300 in locked state, with sheath 1701 held in place. Sheath 1701 is positioned between the internal surface of the guidance element 1220 and the rails 1160 of the first panel 1100, and in some embodiments a top surface of panel 1100, creating a friction fit. The locked state is achieved by bringing the first panel 1100 and second panel 1200 together, typically by rotating the second panel 1200 about the hinges 1302 until it is parallel with the first panel 1100. The locking elements 1140 of the first panel 1100 engage with the receiving elements 1240 of the second panel 1200, securing the guidance device 1300 in a locked position and locking the catheter into place via friction fit.

In some embodiments, the second panel 1200 is removably coupled to the first panel 1100 about the hinges 1302. The hinges 1302 are configured to allow rotation of the second panel 1200 relative to the first panel 1100 to a predetermined position, such as, for example, when the first panel 1100 and second panel 1200 are aligned at approximately 90 degrees to one another. When in this predetermined position, the hinges 1302 are configured to allow disengagement of the second panel 1200 from the first panel 1100, as illustrated in FIGS. 18C and 18D. The hinges 1302 may incorporate one or more cutouts or recesses that align with a spoke (e.g., central spoke) or protrusion of the hinge when in the predetermined orientation. This alignment of the cutouts with the central spoke is configured to allow for the separation of the first panel 1100 and second panel 1200, thereby facilitating removal of the guidance device 1300 from a medical device, such sheath 1701, while the medical device remains secured within the patient, as depicted in FIG. 18E.

In some embodiments, as depicted in FIGS. 18F-18J, the removable coupling of the second panel 1200 to the first panel 1100 about the hinges 1302 allows the guidance device 1300 to be utilized in securing a medical device, such as a sheath 1701, that has been placed into the patient prior to the adherence of the guidance device 1300 to the patient's skin. As shown in FIG. 18F, the first panel 1100 of the guidance device 1300 is configured to be placed in proximity to the entry point of the previously inserted medical device (e.g., sheath 1701 as shown in FIGS. 18F-18J). Subsequently, as illustrated in FIG. 18G, the medical device is positioned within the pathway formed between the two rails 1160 on the first panel 1100. The second panel 1200 is then aligned with the first panel 1100 by way of the hinges 1302, as depicted in FIGS. 18H and 181, where second panel 1200 is connected to first panel 1100 in FIG. 181. Once aligned, the second panel 1200 is rotated about the hinges 1302 to engage with the first panel 1100 in a locked configuration, thereby securing the medical device via a friction fit, as previously described and as shown in FIG. 18J.

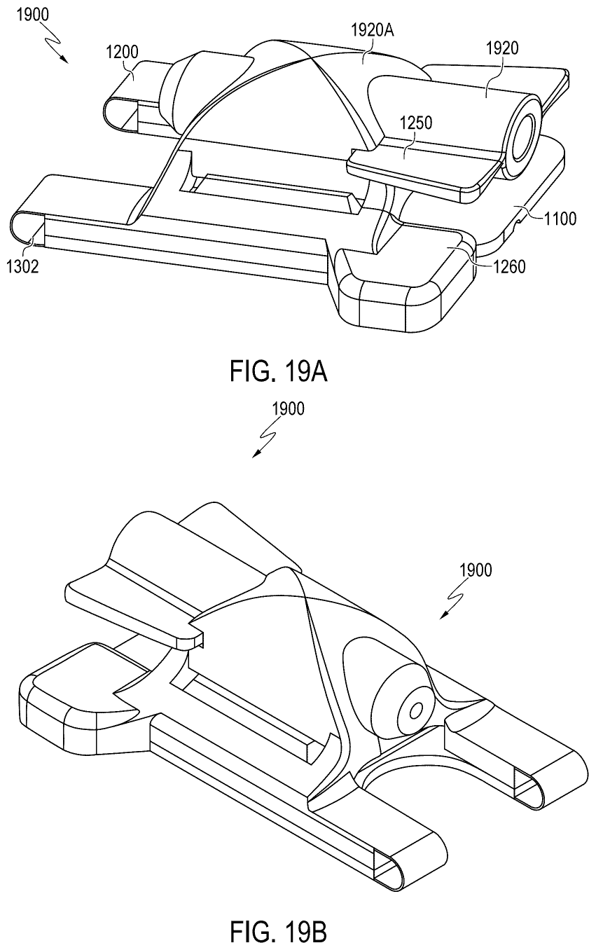

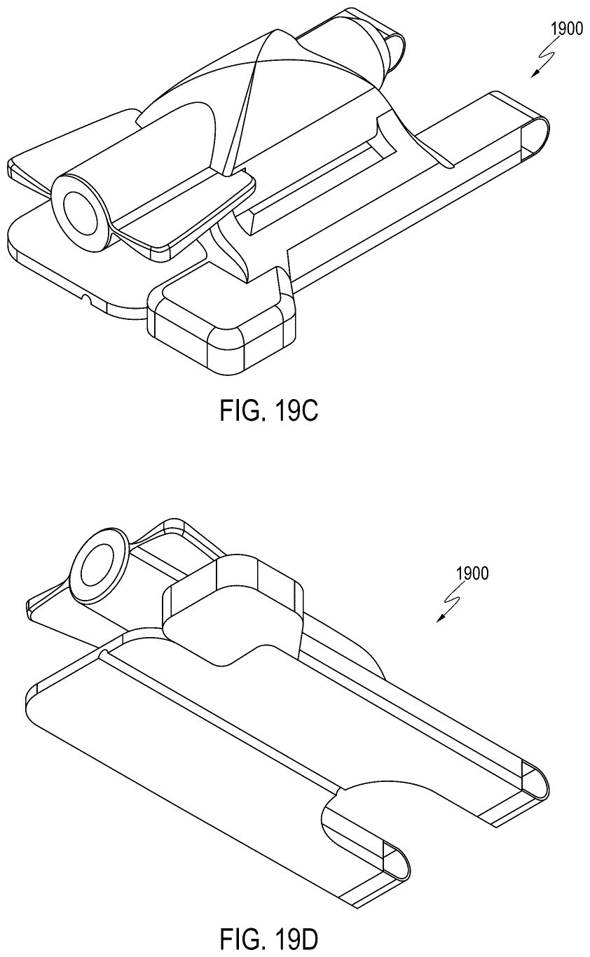

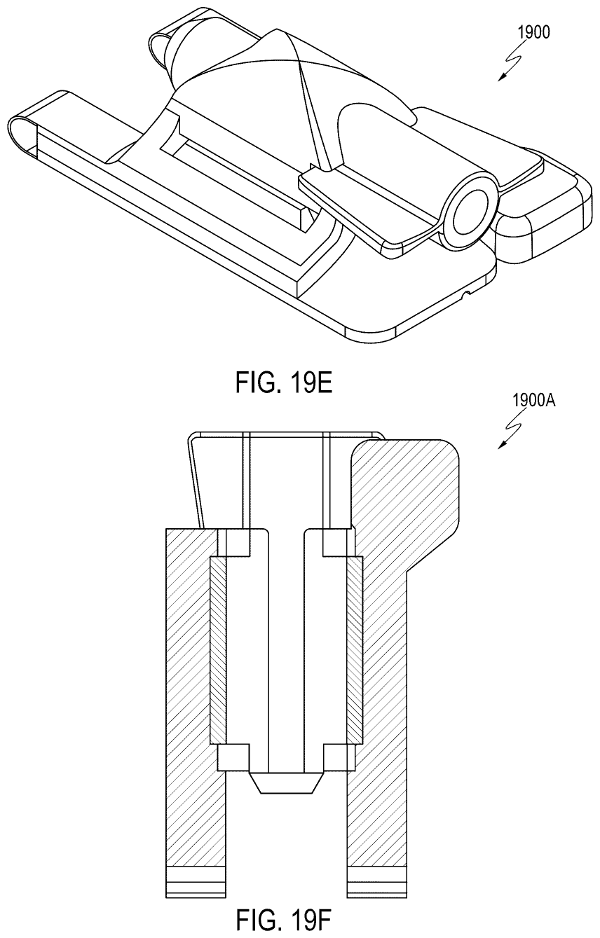

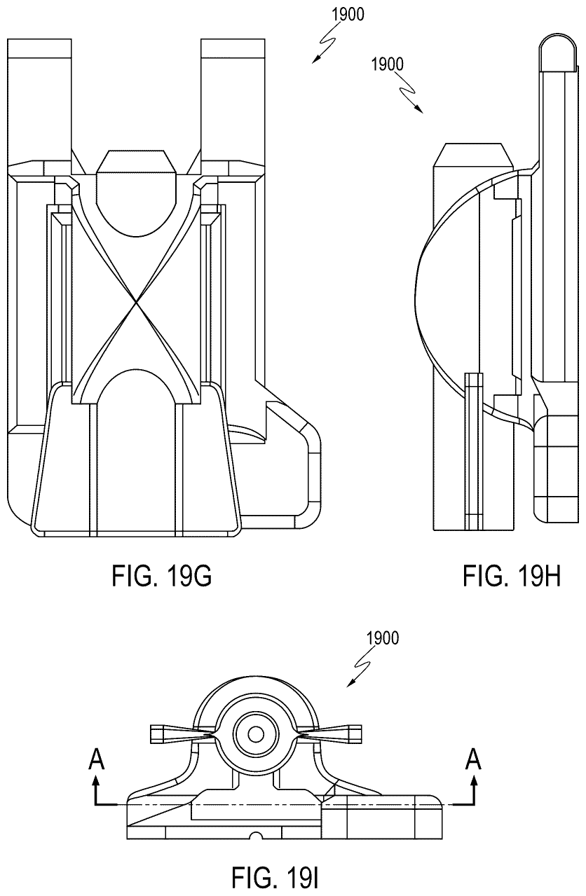

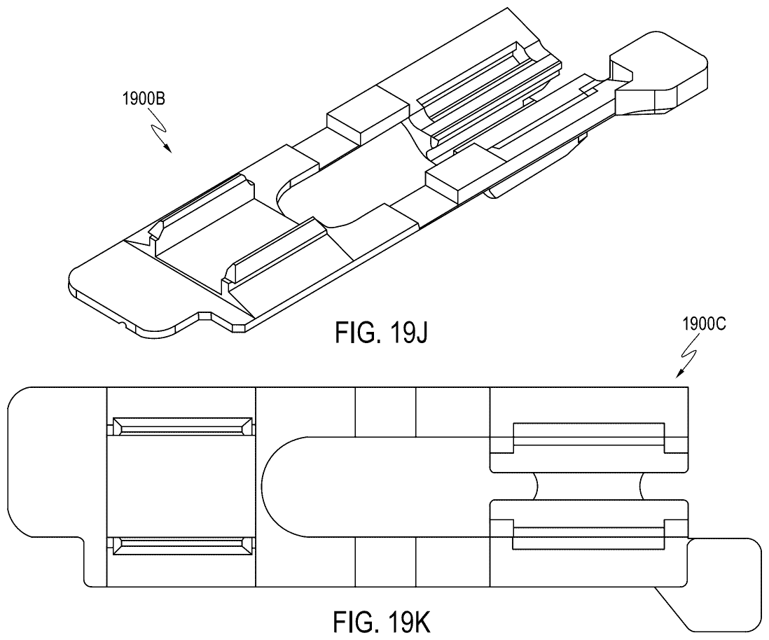

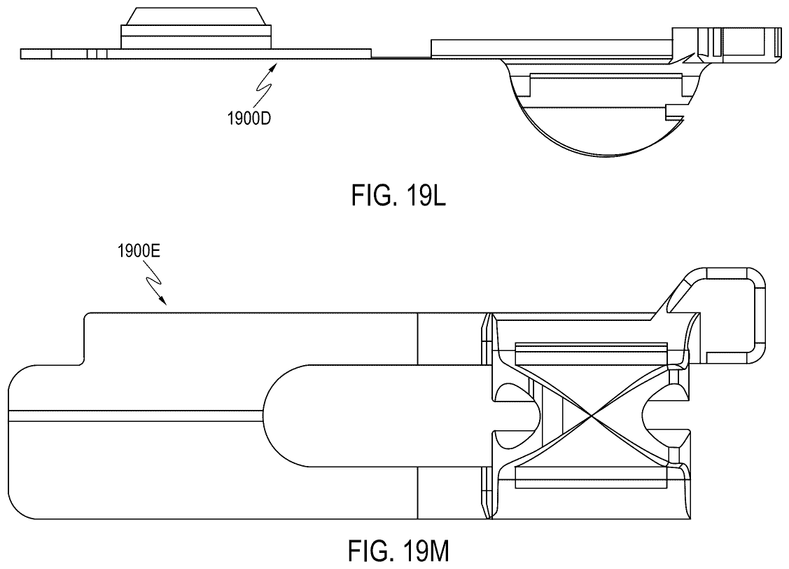

FIGS. 19A-19L depict an alternative embodiment of a guidance device which include similarities and difference relative to the guidance device 100 and guidance device 1300. This embodiment of FIGS. 19A-19L shares some features and principles with the previously described guidance devices, while incorporating distinct elements and methodologies. In this embodiment, as shown in FIG. 19A, a guidance device 1900 includes a guidance element 1920 that incorporates one or more protrusions 1250 located near the openings of guidance element 1920. These protrusions 1250 are designed to accommodate and secure guidance element 1920 and/or one or more components of a medical assembly or medical device. For instance, they may be shaped to receive the wings of a butterfly needle, providing a stable and secure fit for the device. Additionally, the protrusions 1250 may be configured to engage with one or more projections or flanges associated with collar 1230 (shown in FIG. 14). This interaction between the protrusions 1250 and the collar 1230 can help to ensure proper alignment and positioning of the collar within the guidance element 1920, further enhancing the stability and precision of the medical device placement. Guidance element 1920 may be covered by a cover 1920A which may engage with protrusions 1250 to secure guidance element 1920 to second panel 1200.

Moreover, the second panel 1200 of guidance device 1900 may include one or more thumb tabs 1260. These thumb tabs 1260 are positioned to facilitate easy and intuitive manipulation of the second panel 1200 relative to the first panel 1100 throughout the medical procedure. The thumb tabs 1260 provide a tactile and ergonomic point of contact for the user, allowing them to easily grasp and rotate the second panel 1200 as needed. This design element can be particularly beneficial when adjusting the angle of insertion for the medical device or when transitioning the guidance device 1900 between its locked and unlocked states. First panel 1100 and second panel 1200 may be connected via hinges 1302, as discussed herein.

It will be understood that embodiments disclosed in reference guidance device 100 and guidance device 1300 also generally apply to guidance device 1900. For example guidance device 1900 may be used to insert a medical device or component associated with a medical apparatus, as discussed herein.

FIGS. 19B-19L show various views of guidance device 1900. FIG. 19B shows a first perspective view, FIG. 19C shows a second perspective view, FIG. 19D shows a third perspective view, and FIG. 19E shows a fourth perspective view of guidance device 1900. FIG. 19F shows a sectional view 1900A of guidance device 1900, where the section is a plane indicated via line A-A in FIG. 19I. FIG. 19G shows a top view with example approximate dimensions of guidance device 1900. FIG. 19H shows a side view with example approximate dimensions of guidance device 1900. FIG. 19I shows a front view with example approximate dimensions of guidance device 1900. FIGS. 19J-19M show views of guidance device 1900 (shown in FIG. 19A) in an open position. FIG. 19J shows a perspective view 1900B of guidance device 1900 rotated about its hinges. FIG. 19K shows a top view 1900C of guidance device 1900 rotated about its hinges. FIG. 19L shows a side view 1900D of guidance device 1900 rotated about its hinges. FIG. 19M shows a bottom view 1900E of guidance device 1900 rotated about its hinges.

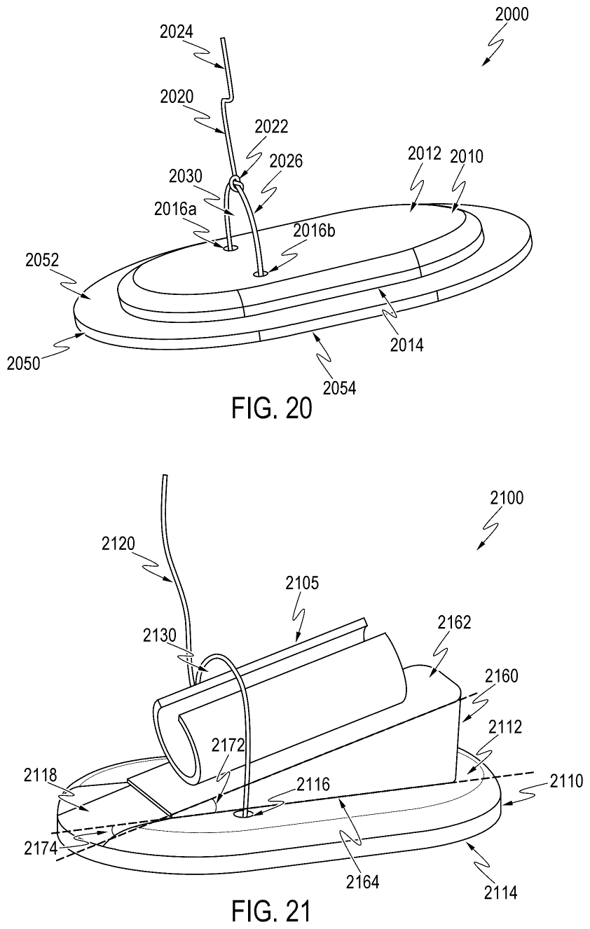

In some embodiments, an alternative configuration of the guidance device discussed herein is provided, as illustrated in FIG. 20. This embodiment shares some features and principles with the previously described guidance devices, while incorporating distinct elements and methodologies. A guidance device 2000, as shown in FIG. 20 may include a panel 2010 and a fastener 2020.

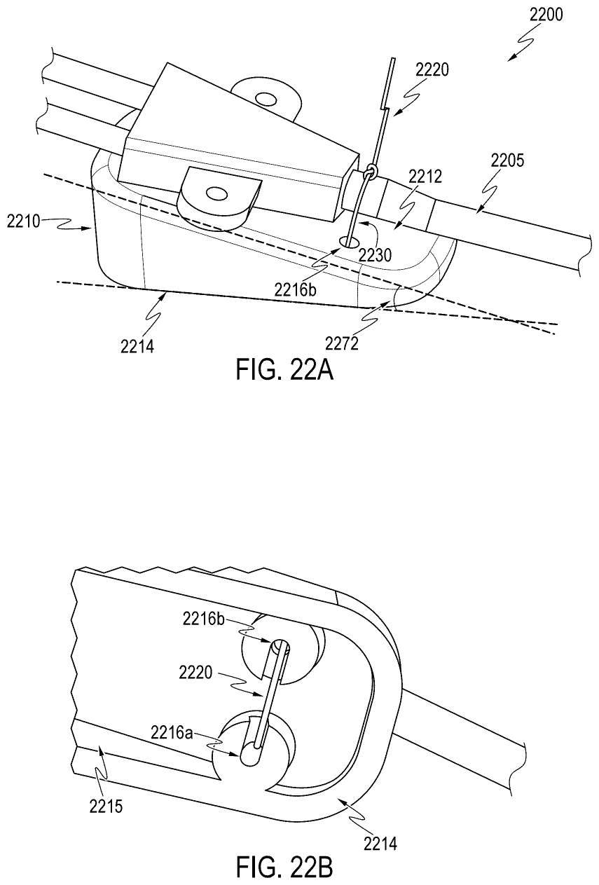

In some embodiments, the panel 2010 may include a first surface 2012 and a second surface 2014 opposite the first surface 2012, and a plurality of holes formed on the first surface 2012. In some embodiments, the first surface 2012 of the panel 2010 and the second surface 2014 of the panel 2010 may be substantially parallel to each other as shown in FIG. 20. In other embodiments, the first surface 2012 of the panel 2010 and the second surface 2014 of the panel 2010 may be non-parallel to each other (e.g., as shown in FIG. 22A, further discussed herein).

The plurality of holes may be formed on the first surface 2012 of the panel 2010 to receive the fastener 2020. In some embodiments, the plurality of holes may include a first hole 2016a and a second hole 2016b. The fastener 2020 may be configured to pass through the first hole 2016a and the second hole 2016b, and form a loop 2030. The loop 2030 formed by the fastener 2020 may be defined by the fastener 2020 and the first surface 2012 of the panel 2010. The loop 2030 may be configured to receive a medical device between the fastener 2020 and the first surface 2012 of the panel. The size of the loop 2030 may be adjustable to receive the medical device and secure the medical device. For example, the loop 2030 may be loosened to receive the medical device and fastened to secure the medical device to the panel 2010.

In some embodiments, the plurality of holes may extend from the first surface 2012 to the second surface 2014. In other embodiments, the panel 2010 may include an internal hollow space, and the plurality of holes may extend from the first surface 2012 to the internal hollow space. It will be understood that the space between two or more holes may be any applicable percentage of available space across a length, width, or depth of the first surface. For example, the space between two or more holes may be determined based on the size of a component (e.g., a medical device) to be inserted through a loop created by the two or more holes and the corresponding fasteners.

Although there are only two holes and one fastener shown in FIG. 20, in some embodiments, the guidance device 2000 may include more or less than two holes (e.g., 1, 3, 4, 5, 6, 7, 8, 9, 10) in the panel 2010 and more than one fastener (e.g., 2, 3, 4, 5, 6, 7, 8, 9, 10). For example, in some embodiments, the guidance device 2000 may further include a third hole and a fourth hole in the panel and a second fastener. The second fastener may be configured to pass through the third hole and the fourth hole, and form a second loop. In some embodiments, the first loop and the second loop may be aligned along a length direction or a direction perpendicular to the length direction of the panel 2010 without overlapping each other. In other embodiments, the first loop and the second loop may have any other suitable alignments (e.g., overlapping alignment). The second loop formed by the second fastener may be defined by the second fastener and the first surface 2012 of the panel 2010. The second loop may be configured to receive a medical device between the second fastener and the first surface 2012 of the panel 2010. The size of the second loop may be adjustable to receive the medical device and secure the medical device.

In some embodiments, the fastener 2020 may be a wire, a suture, a string, and/or a strand. In other embodiments, the fastener 2020 may be any other suitable line/cord/thread/cable that can receive and/or secure a medical device.

In some embodiments, the medical device may include a needle and/or a catheter (e.g., any previously discussed needles and/or catheters). In other embodiments, the medical device may include any other suitable device intended for medical use (e.g., guidewire or any other guidance element).

In some embodiments, the loop 2030 may be formed by the fastener 2020 being in a knotted configuration 2022 as shown in FIG. 20. The knotted configuration 2022 may be at least one of a slip knot, an overhand knot, a bowline knot, a figure eight knot, or a taut-line hitch. In other embodiments, the loop 2030 may be formed by any other suitable configuration (e.g., simply one way knot requiring a second knot to lock, or more complicated knot that does not require a second knot). In some embodiments, a portion of the fastener 2020 may include a feature (e.g., teeth) and/or locking mechanism as in a zip tie to form the loop 2030.

In some embodiments, the size of the loop 2030 may be adjusted by pulling a portion of the fastener 2020. For example, the size of the loop 2030 may be decreased by pulling a free end portion 2024 of the fastener 2020 away from the loop 2030 (e.g., while holding the knot configuration/locking mechanism). The size of the loop 2030 may be increased by pulling a portion 2026 of the fastener 2020 forming the loop 2030 away from the free end portion 2024 (e.g., while holding the knot configuration/locking mechanism).

In some embodiments, the guidance device 2000 may further include an attachment element 2050 on the second surface 2014 of the panel 2010. The attachment element 2050 may include a dual-sided adhesive. A first side 2052 of the dual-sided adhesive may be adhered to the second surface 2014 of the panel 2010, and a second side 2054 of the dual-sided adhesive may be configured to adhere to an external surface, such as the skin of a patient. In some other embodiments, the second surface 2014 of the panel 2010 itself may form or have an adhesive surface.

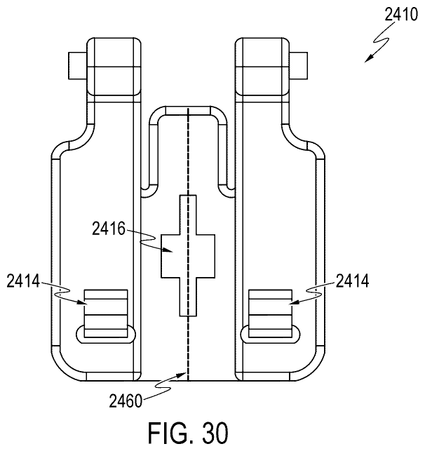

In some embodiments, the guidance device 2000 may further include a longitudinal guiding line (e.g., on the first surface 2012 and/or a second surface 2014 of the panel 2010). The longitudinal guiding line may be configured to show an orientation of the medical device, such as a needle or a catheter. In some embodiments, the longitudinal guiding line may be a radiopaque line (e.g., using a radio-opaque marker) and visible under fluoroscopic light or using a fluoroscopy technique. In other embodiments, the longitudinal guiding line may be formed in any other suitable manner (e.g., a line in a color different from the color of the first panel 2410). In some embodiments, the longitudinal guiding line may be formed on a top or bottom surface of the panel 2010. Such a guiding line is further disclosed herein in reference to FIG. 30.

In some embodiments, an alternative configuration of the guidance device discussed herein is provided, as illustrated in FIG. 21. This embodiment shares some features and principles with the previously described guidance devices, while incorporating distinct elements and methodologies. A guidance device 2100, as shown in FIG. 21 may include a panel 2110 and a fastener 2120.

In some embodiments, the panel 2110 may include a first surface 2112 and a second surface 2114 opposite the first surface 2112, and a plurality of holes 2116 formed on the first surface 2112. The plurality of holes 2116 may receive the fastener 2120.

In some embodiments, the plurality of holes 2116 may include a first hole and a second hole. The fastener 2120 may be configured to pass through the first hole and the second hole, and form a loop 2130. The loop 2130 formed by the fastener 2120 may be defined by the fastener 2120 and the first surface 2112 of the panel 2110. The loop 2130 may be configured to receive a medical device 2105 between the fastener 2120 and the first surface 2112 of the panel 2110. The size of the loop 2130 may be adjustable to receive the medical device 2105 and secure the medical device 2105. For example, the loop 2130 may be loosened to receive the medical device 2105 and fastened to secure the medical device 2105 to the panel 2110 and/or an angle support 2160, which will be discussed in more detail below.

In some embodiments, the medical device may include a needle and/or a catheter. In other embodiments, the medical device may include any other suitable device intended for medical use (e.g., guidewire or any other guidance element).

In some embodiments, the guidance device 2100 may further include an angle support 2160 disposed on the panel 2110. The angle support 2160 may include a first angle surface 2162 configured to face the medical device 2105, and a second angle surface 2164 opposite the first angle surface 2162 and configured to face the first surface 2112 of the panel 2110.

In some embodiments, a first angle 2172 formed between the first angle surface 2162 and the second angle surface 2164 may be greater than 0° and less than 90°. For example, the first angle 2172 formed between the first angle surface 2162 and the second angle surface 2164 may be in a range of about 1° to about 89°, about 10° to about 80°, about 15° to about 70°, about 20° to about 60°, about 25° to about 50°, or about 30° to about 40°.

In some embodiments, the panel 2110 may further include a slope portion 2118. In some embodiments, a portion of the panel 2110 may be removed to form the slope portion 2118. The slope portion 2118 may be sloped, inclined, and/or angled, for example, relative to a bottom surface of the panel 2110. In some embodiments, a second angle 2174 formed between the slope portion 2118 of the panel 2110 and the second angle surface 2164 may be substantially the same as the first angle 2172 formed between the first angle surface 2162 and the second angle surface 2164. In other embodiments, the second angle 2174 formed between the slope portion 2118 of the panel 2110 and the second angle surface 2164 may be different from the first angle 2172 formed between the first angle surface 2162 and the second angle surface 2164.

In some embodiments, a width of the slope portion 2118 may be the same as or similar to a width of the angle support 2160. In other embodiments, the width of the slope portion 2118 may be different from the width of the angle support 2160.

Other configurations, features, or characteristics of the guidance device 2100 (e.g., types of fastener, holes, loop formation, loop size adjustment, attachment element, adhesive surface) may be similar to and/or same as the ones described above with respect to the guidance device 2000.

In some embodiments, an alternative configuration of the guidance device discussed herein is provided, as illustrated in FIGS. 22A and 22B. This embodiment shares some features and principles with the previously described guidance devices, while incorporating distinct elements and methodologies. A guidance device 2200, as shown in FIG. 22A may include a panel 2210 and a fastener 2220.

In some embodiments, the panel 2210 may include a first surface 2212 and a second surface 2214 opposite the first surface 2212, and a plurality of holes formed on the first surface 2212. The plurality of holes may receive the fastener 2220.

In some embodiments, the first surface 2212 of the panel 2210 and the second surface 2214 of the panel 2210 may be non-parallel to each other. In some embodiments, an angle 2272 formed between the first surface 2212 and the second surface 2214 may be greater than 0° and less than 90°. For example, the angle 2272 may be in a range of about 1° to about 89°, about 10° to about 80°, about 15° to about 70°, about 20° to about 60°, about 25° to about 50°, or about 30° to about 40°.

The plurality of holes may be formed on the first surface 2212 of the panel 2210 to receive the fastener 2220. In some embodiments, the plurality of holes may include a first hole 2216a and a second hole 2016b, as shown in FIGS. 22A and 22B. The fastener 2220 may be configured to pass through the first hole 2216a and the second hole 2216b, and form a loop 2230. The loop 2230 formed by the fastener 2220 may be defined by the fastener 2220 and the first surface 2212 of the panel 2210. The loop 2230 may be configured to receive a medical device 2205 between the fastener 2220 and the first surface 2212 of the panel 2210. The size of the loop 2230 may be adjustable to receive the medical device 2205 and secure the medical device 2205. For example, the loop 2230 may be loosened to receive the medical device 2205 and fastened to secure the medical device 2205 to the panel 2210.

In some embodiments, the plurality of holes may extend from the first surface 2212 to the second surface 2214. In other embodiments, the panel 2210 may include an internal hollow space 2215, and the plurality of holes may extend from the first surface 2212 to the internal hollow space 2215.

Other configurations, features, or characteristics of the guidance device 2200 (e.g., types of fastener, holes, loop formation, loop size adjustment, attachment element, adhesive surface) may be similar to and/or same as the ones described above with respect to the guidance device 2000 and/or 2100.

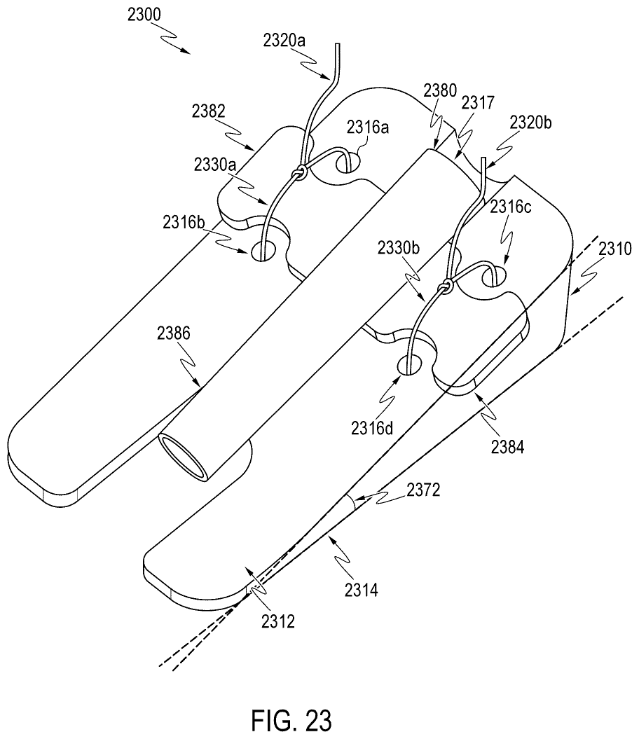

In some embodiments, an alternative configuration of the guidance device discussed herein is provided, as illustrated in FIG. 23. This embodiment shares some features and principles with the previously described guidance devices, while incorporating distinct elements and methodologies. A guidance device 2300, as shown in FIG. 23 may include a panel 2310 and one or more fasteners.

In some embodiments, the panel 2310 may include a first surface 2312 and a second surface 2314 opposite the first surface 2312, and a plurality of holes formed on the first surface 2312. In some embodiments, the first surface 2312 of the panel 2310 and the second surface 2314 of the panel 2310 may be non-parallel to each other. An angle 2372 formed between the first surface 2312 of the panel 2310 and the second surface 2314 of the panel 2310 is greater than 0° and less than 90°. For example, the angle 2372 may be in a range of about 1° to about 89°, about 10° to about 80°, about 15° to about 70°, about 20° to about 60°, about 25° to about 50°, or about 30° to about 40°. In other embodiments, the first surface 2312 of the panel 2310 and the second surface 2314 of the panel 2310 may be substantially parallel to each other.

The plurality of holes may be formed on the first surface 2312 of the panel 2310 to receive the one or more fasteners. In some embodiments, the plurality of holes may include a first hole 2316a, a second hole 2316b, a third hole 2316c, and a fourth hole 2316d.

In some embodiments, the one or more fasteners may include a first fastener 2320a and a second fastener 2320b. The first fastener 2320a may pass through the first hole 2316a and the second hole 2316b, and form a first loop 2330a. The second fastener 2320b may pass through the third hole 2316c and the fourth hole 2316d, and form a second loop 2330b.

Although there are only four holes and two fasteners shown in FIG. 23, in some embodiments, the guidance device 2300 may include more or less than four holes (e.g., 1, 2, 3, 5, 6, 7, 8, 9, 10) in the panel 2310 and more or less than two fasteners (e.g., 1, 3, 4, 5, 6, 7, 8, 9, 10).

The first loop 2330a formed by the first fastener 2320a may be defined by the first fastener 2320a and the first surface 2312 of the panel 2310. In some embodiments, the first loop 2330a formed by the first fastener 2320a may be configured to receive a first device 2380. The size of the first loop 2330a may be adjustable to receive the first device 2380 and secure the first device 2380. For example, the first loop 2330a may be loosened to receive the first device 2380 and fastened to secure the first device 2380 to the panel 2310.

The second loop 2330b formed by the second fastener 2320b may be defined by the second fastener 2320b and the first surface 2312 of the panel 2310. In some embodiments, the second loop 2330b formed by the second fastener 2320b may be configured to receive a first device 2380. The size of the second loop 2330b may be adjustable to receive the first device 2380 and secure the first device 2380. For example, the second loop 2330b may be loosened to receive the first device 2380 and fastened to secure the first device 2380 to the panel 2310.

In some embodiments, the first device 2380 may include a medical device (e.g., a needle and/or a catheter) and/or a guidance element configured to guide a medical device. In other embodiments, the first device 2380 may include any other suitable device intended for medical use.

In some embodiments, the first device 2380 may include a first wing 2382; a second wing 2384, and a guiding portion 2386 disposed between the first wing 2382 and the second wing 2384. In this case, the first device 2380 may serve as a guidance element, and the guiding portion 2386 of the first device 2380 may be configured to receive a medical device. For example, the guiding portion 2386 may include an internal hollow space configured to receive a medical device. In some embodiments, the guiding portion 2386 may be in an elongated tube/pipe shape. In other embodiments, the guiding portion 2386 may be in any other suitable shape.

In some embodiments, the first wing 2382 may be configured to be secured to the panel 2310 by the first loop 2330a. Similarly, the second wing 2384 may be configured to be secured to the panel 2310 by the second loop 2330b.

In some embodiments, the panel 2310 may include a groove 2317 configured to receive the guiding portion 2386 of the first device 2380. In some embodiments, the groove 2317 may be formed in a central portion of the panel 2310. In other embodiments, the groove 2317 may be formed in any other suitable portion of the panel 2310 (e.g., left or right portion of the panel 2310).

In some embodiments, the first loop 2330a formed by the first fastener 2320a may be configured to receive a first device, and the second loop 2330b formed by the second fastener 2320b may be configured to receive a second device different from the first device. A size of the first loop 2330a may be adjustable to receive and/or secure the first device, and a size of the second loop 2330b may be adjustable to receive and/or secure the second device. The first/second device may include a medical device (e.g., a needle and/or a catheter) and/or a guidance element configured to guide a medical device. In other embodiments, the first/second device may include any other suitable device intended for medical use.

Other configurations, features, or characteristics of the guidance device 2300 (e.g., types of fasteners, holes, loop formation, loop size adjustment, attachment element, adhesive surface) may be similar to and/or same as the ones described above with respect to the guidance device 2000, 2100, and/or 2200 and, thus, duplicate description may be omitted.

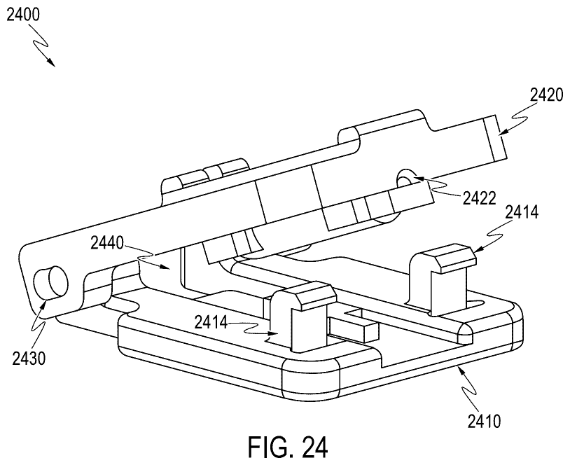

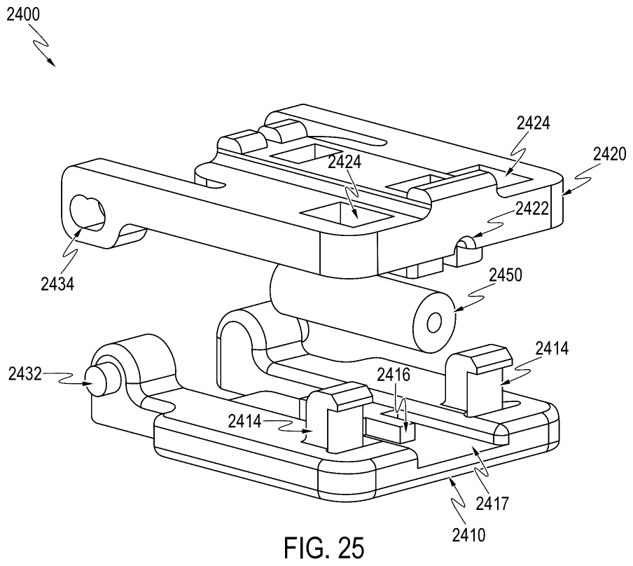

In some embodiments, an alternative configuration of the guidance device discussed herein is provided, as illustrated in FIGS. 24-32. This embodiment shares some features and principles with the previously described guidance devices, while incorporating distinct elements and methodologies. A guidance device 2400, as shown in FIGS. 24-32, may include a first panel 2410 and a second panel 2420. The first panel 2410 and the second panel 2420 may be connected by one or more hinges 2430, and may each include a portion of a cutout defining a target region 2440. Hinges 2430 may include hinge holes 2434 that are shaped to allow rotation and sliding of hinge protrusion 2432, as further discussed herein in reference to FIG. 25.

The hinges 2430 may be positioned on opposing sides of the target region 2440, such that the first panel 2410, second panel 2420, and opposing hinges 2430 define at least a portion of target region 2440. The guidance device 2400 is operable to position a medical apparatus relative to a portion of the target region 2440 and adjust one or more angle of the medical apparatus by rotating the second panel 2420 relative to the first panel 2410 about the hinges 2430 to position the medical apparatus at a desired angle.

To further assist with alignment of the medical apparatus to the target region 2440, one of the first panel 2410 or second panel 2420 may be attached (e.g., adhere) to a first surface (e.g., a patient surface), such as being secured (e.g., via an adhesive) to the skin of a patient. The guidance device 2400 may be configured in either an unlocked configuration or a locked configuration, and may be movable between the two configurations. In the unlocked configuration, the guidance device 2400 may be configured so that first panel 2410 and second panel 2420 are connected to and/or in contact with one another only by way of hinges 2430, and second panel 2420 is free to rotate relative to first panel 2410. In the locked configuration, the second panel 2420 is positioned so one or more surface and/or element of the second panel engages and/or interacts with one or more surface and/or element of the first panel 2410 such that the first panel 2410 and second panel 2420 are locked in place relative to one another and unable to rotate relative to one another about hinges 2430.

The construction material of the guidance device 2400, particularly for the first panel 2410 and the second panel 2420, may be chosen based on a desired balance of flexibility and rigidity and may be or may include a type of plastic, rubber, and/or the like. The chosen material may allow the panels to conform subtly to the contours of a patient's skin, thereby enhancing comfort and fit, while simultaneously retaining sufficient rigidity to facilitate accurate alignment and secure positioning of a medical apparatus, such as needle or catheter. Alternative materials may also be considered, provided they meet the conformability to skin contours and maintenance of structural integrity for device alignment.

In some embodiments, the first panel 2410 includes one or more locking elements 2414, which, in some embodiments, may be referred to as first or second engagement elements. These locking elements 2414 may be situated on a surface of the first panel 2410 facing the second panel 2420, such as the top surface of the first panel 2410, as illustrated in FIGS. 24-26. The locking elements 2414 can be located at various positions along the surface of the first panel 2410. The design of each locking element 2414 may incorporate a protrusion or extension that is configured to engage with the receiving elements 2424 located on the second panel 2420, which, in some embodiments, may be referred to as first or second engagement elements. It will be understood that locking elements, such as locking elements 2414 may, instead, be positioned on the second panel 2420 and receiving elements, such as receiving elements 2424, may be positioned on the first panel 2410.

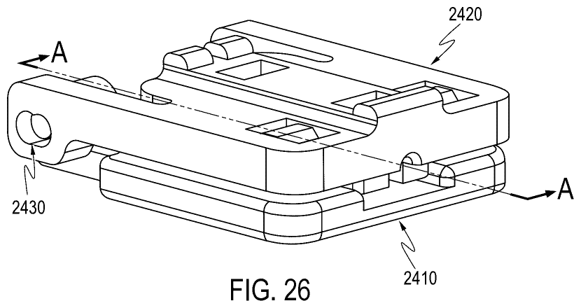

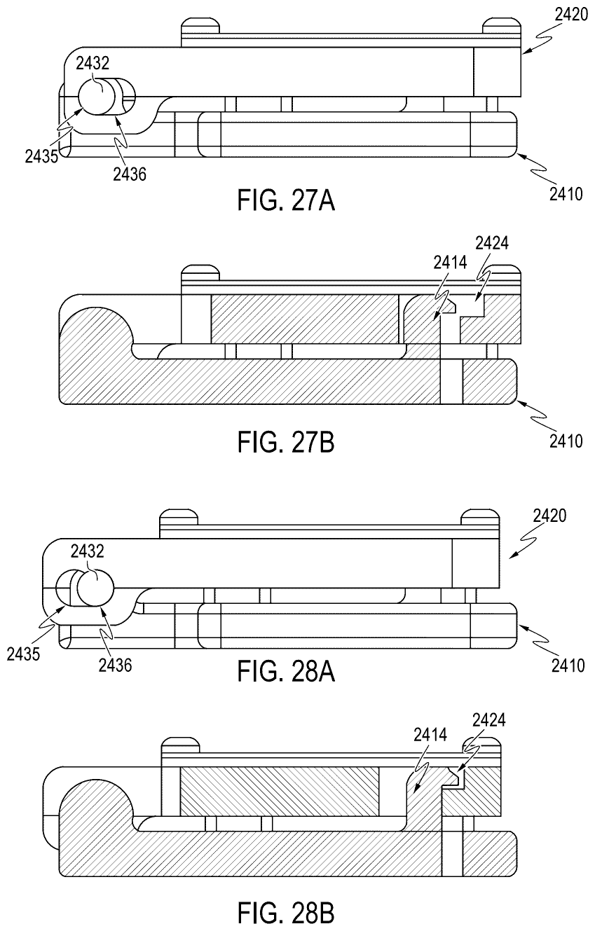

In some embodiments, the hinge 2430 may be formed by hinge protrusions 2432 of one panel (e.g., the first panel 2410) and hinge holes 2434 of the other panel (e.g., second panel 2420), as shown in FIG. 25. For simplicity of discussion, hinge protrusions 2432 are further discussed herein as being part of the first panel 2410 and hinge holes 2434 are discussed herein as being part of the second panel 2420. FIG. 27A illustrates a side view of the guidance device 2400 when the guidance device 2400 is in an unlocked configuration. FIG. 27B depicts a cross-sectional view of the guidance device 2400 along the line A-A of FIG. 26 when the guidance device 2400 is in an unlocked configuration. As shown in FIG. 27A, the hinge hole 2434 may include a first portion 2435 and a second portion 2436. When the hinge protrusion 2432 of the first panel 2410 is disposed in the first portion 2435, the guidance device 2400 may be in an unlocked configuration. In this case, the locking elements 2414 may not be engaged with the receiving elements 2424 as shown in FIG. 27B.

FIG. 28A illustrates a side view of the guidance device 2400 when the guidance device 2400 is in a locked configuration. FIG. 28B depicts a cross-sectional view of the guidance device 2400 along the line A-A of FIG. 26 when the guidance device 2400 is in a locked configuration. When the hinge protrusion 2432 of the first panel 2410 is disposed in the second portion 2436, the guidance device 2400 may be in a locked configuration. In this case, the locking elements 2414 may be engaged with the receiving elements 2424 as shown in FIG. 28B.

In this way, aspects of the present disclosure allow the guidance device 2400 to transition between the locked configuration and the unlocked configuration by sliding the hinge protrusion 2432 within the hinge hole 2434 between the first portion 2435 and the second portion 2436. The sliding direction of the hinge protrusion 2432 within the hinge hole 2434 may be substantially parallel to an external surface (e.g., skin of a patient) on which the guidance device 2400 is configured to be placed. This structure or configuration of the guidance device 2400 may ease the locking and unlocking process even when the guidance device 2400 is placed on a soft tissue where it is difficult to apply force in a direction perpendicular to the soft tissue.

When the guidance device 2400 transitions from an unlocked configuration to a locked configuration, the locking elements 2414 interlock with the receiving elements 2424, thereby inhibiting relative movement between the first panel 2410 and the second panel 2420. It will be appreciated that the relative positioning of the locking elements 2414 and the receiving element 2424 are shown as being associated with the first and second panels, respectively, but that the guidance device 2400 is operable with locking elements 2414 positioned on the second panel 2420 and the receiving elements 2424 positioned on the first panel.

In some embodiments, the first panel 2410 may include one or more friction members 2416, as shown in FIG. 25, which may be positioned along the midline of a panel such as first panel 2410. The one or more frictional members may be circumferential, or any other applicable shape (e.g., a shape that corresponds to guidance element 2422, as discussed herein). One or more friction members 2416 may be integrally formed with the first panel 2410 or separately attached, and a portion of it may protrude above the top surface of the first panel 2410. The construction of the friction member 2416 can vary; it may be made from the same material as the first panel 2410, ensuring uniformity, or from a different material, selected to enhance specific characteristics like durability or frictional properties. The material of the friction member 2416 may be chosen for its rigidity or its capacity to be resiliently deformable. In scenarios where it is resiliently deformable, the friction member 2416 is designed to deform under a compression force, where the one or more friction member 2416 exerts a reactive force that provides a friction coupling between the one or more friction member 2416 and the outside object which is applying the compression force.

The first panel 2410 may further include one or more depressed portions 2417, as shown in FIG. 25. The one or more depressed portions 2417 may manifest as cutouts that either extend entirely through a depth of the panel or only partially. The one or more depressed portions 2417 are, in some embodiments, positioned along the midline of the panel, adjacent to or interspersed among one or more friction members 2416.

In some embodiments, the second panel 2420 may include a guidance element 2422, as shown in FIG. 25. This guidance element 2422 may be positioned in the middle portion of the second panel 2420. Guidance element 2422 may receive one or more medical devices or guiding member 2450, as further discussed herein. The design, shape, and/or positioning of the guidance element 2422 may be such that it can accommodate medical devices like needles, catheters, etc., facilitating their insertion and positioning during medical procedures. The guidance element 2422 may be shaped or configured in various ways to ensure compatibility with different types and sizes of medical devices. The guidance element 2422, as part of the second panel 2420, may be generally semi-cylindrical in shape. The semi-cylindrical shape of the guidance element 2422 provides a structured pathway for guiding medical devices. It will be understood that guidance element 2422 may have any other applicable shape that allows guidance element 2422 to receive, position, and/or secure a medical device.

In some embodiments, the guidance device 2400 may further include a guiding member 2450. The guiding member 2450 may include an internal channel (e.g., a lumen) configured to receive a medical device, such as a needle, catheter, etc. In some embodiments, the guiding member 2450 may be separate from the first panel 2410 and the second panel 2420. In some embodiments, the guiding member 2450 may be connected to the second panel 2420, for example, by a friction fit. In other embodiments, the guiding member 2450 may be connected to the first panel 2420, for example, by a friction fit. In other embodiments, the guiding member 2450 may be part of the first panel 2410 and/or the second panel 2420.

In some embodiments, the guiding member 2450 may be in a tube-shape. In other embodiments, the guiding member 2450 may have any other suitable shape (e.g., rectangular prism, hexagonal prism, etc.). In some embodiments, the guiding member 2450 may be made of soft materials, such as silicone, Pebax, polyurethane, and rubber. In other embodiments, the guiding member 2450 may be made of any other suitable materials.

When the first panel 2410 and the second panel 2420 are fastened together, the guiding member 2450 may be compressed between the first panel 2410 and the second panel 2420, thereby securing the guiding member 2450 and a medical device positioned in the guiding member 2450. Aspects of the present disclosure may allow the first panel 2410 and the second panel 2420 to be unfastened without disturbing the medical device while holding the medical device within the guiding member 2450.

In some embodiments, when a medical device, such as needle or catheter, is interfaced with the guidance element 2422 and/or the guiding member 2450, the friction member 2416 may further restrict the movement of the medical device, which may ensure that the medical device, such as a catheter, is securely held in place, reducing the likelihood of displacement during and/or after a medical procedure.

In some embodiments, the guiding member 2450 may include a longitudinal slit configured to receive a medical device. In this case, the guiding member 2450 can be removed from the medical device (after the first panel 2410 and the second panel 2420 are unfastened) without disturbing the medical device.

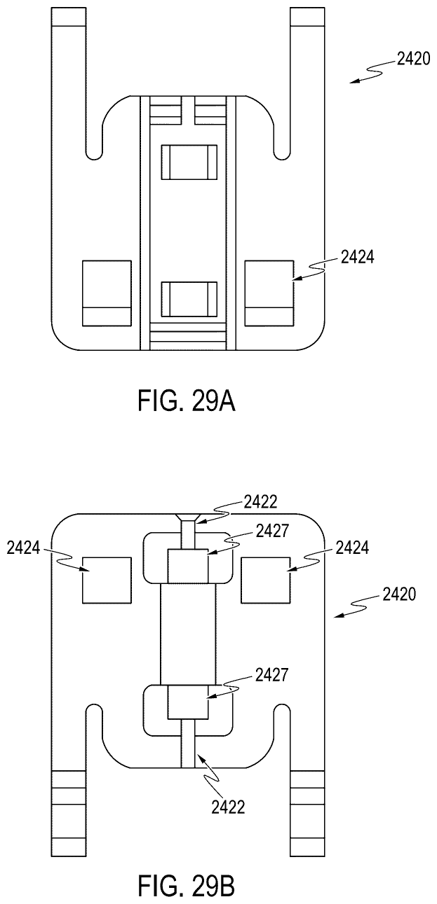

In some embodiments, the second panel 2420 may include one or more guiding member holders 2427, as shown in FIG. 29B. The guiding one or more guiding member holders 2427 may be a recess in the second panel 2420, and configured to receive the guiding member 2450. The one or more guiding member holders 2427 may be connected to the one or more guidance elements 2422.

As previously discussed, the second panel 2420 may also include one or more receiving elements 2424 (e.g., as shown in FIG. 29A), which have been previously mentioned in the context of their interaction with the locking elements 2414 of the first panel 2410. These receiving elements 2424 may be shaped to engage with the locking elements 2414 when the guidance device 2400 transitions into its locked configuration. This engagement ensures that the first panel 2410 and the second panel 2420 are secured relative to each other, preventing unintentional movement.

In some embodiments, hinges 2430 serve as the connecting mechanism between the first panel 2410 and the second panel 2420 in the guidance device 2400. As illustrated in FIGS. 24-32, there may be two hinges, which may be positioned on opposing sides of the target region 2440. These hinges 2430 may allow for the relative movement of the first panel 2410 and second panel 2420, allowing them to be adjusted as needed for the specific requirements of a medical procedure. The placement of the hinges 2430 is such that they may facilitate the opening and closing motion of the panels while maintaining the alignment of the target region 2440. This configuration allows for the flexible positioning of the first panel 2410 and second panel 2420 while ensuring they remain interconnected throughout the operation of the guidance device 2400. Although two hinges 2430 are shown in FIGS. 24-32 and generally discussed herein, it will be understood that any applicable number (e.g., one or more) of hinges 2430 may be used to implement the systems and methods disclosed herein.

Referring to FIG. 30, in some embodiments, a top surface of the first panel 2410 may include a longitudinal guiding line 2460. The longitudinal guiding line 2460 may be configured to show an intended orientation of the medical device, such as a needle or a catheter. In some embodiments, the longitudinal guiding line 2460 may be a radiopaque line (e.g., using a radio-opaque marker) and visible under fluoroscopic light or fluoroscopy. In other embodiments, the longitudinal guiding line 2460 may be formed in any other suitable manner (e.g., a line in a color different from the color of the first panel 2410). In some embodiments, the longitudinal guiding line 2460 may be formed on a bottom surface of the first panel 2410 and/or a top or bottom surface of the second panel 2420.

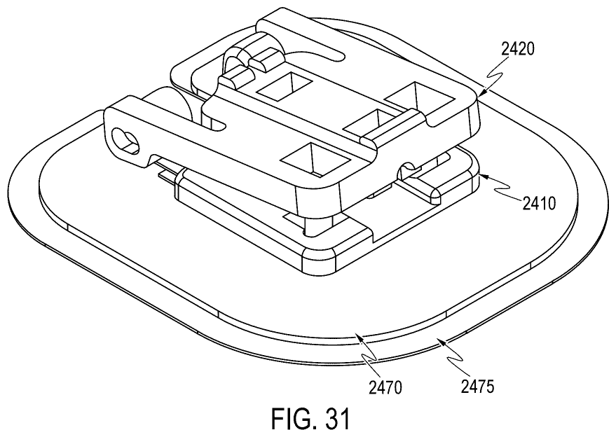

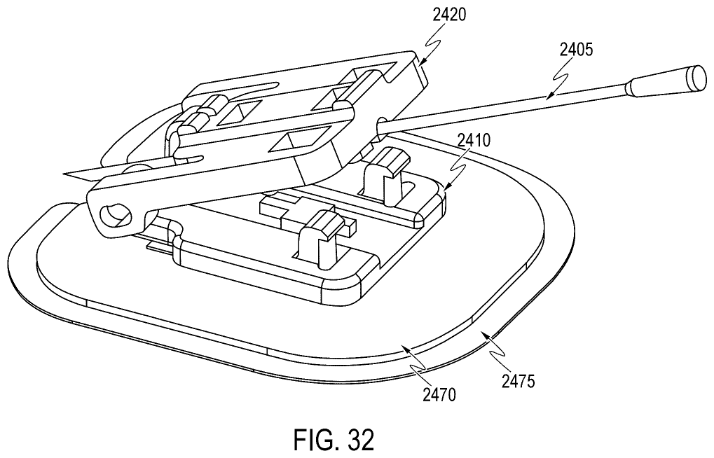

Referring to FIGS. 31-32, in some embodiments, the guidance device 2400 may further include an attachment base 2470 attached to a bottom surface of the first panel 2410. The attachment base 2470 may include a dual-sided adhesive. For example, a first side of the dual-sided adhesive may be adhered to the bottom surface of the first panel 2410, and a second side of the dual-sided adhesive may be configured to adhere to an external surface 2475, such as the skin of a patient. In some other embodiments, the second surface of the first panel 2410 itself may form or have an adhesive surface. Such an attachment of guidance device 2400 to a surface (e.g., the skin of the patient) may mitigate or prevent movement of the guidance device 2400 during insertion or use of medical device 2405 (e.g., while guidance device 2400 is in the unlocked configuration shown in FIG. 31 and/or in the locked configuration shown in FIG. 32).

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the disclosure is by way of example, and the scope of the disclosure is not limited to the exact details shown or described.

Although the foregoing detailed description of the present disclosure has been described by reference to an exemplary embodiment, and the best mode contemplated for carrying out the present disclosure has been shown and described, it will be understood that certain changes, modification or variations may be made in embodying the above disclosure, and in the construction thereof, other than those specifically set forth herein, may be achieved by those skilled in the art without departing from the spirit and scope of the disclosure, and that such changes, modification or variations are to be considered as being within the overall scope of the present disclosure. Therefore, it is contemplated to cover the present disclosure and any and all changes, modifications, variations, or equivalents that fall within the true spirit and scope of the underlying principles disclosed and claimed herein. Consequently, the scope of the present disclosure is intended to be limited only by the attached claims, all matter contained in the above description and shown in the accompanying drawings shall be interpreted as illustrative and not in a limiting sense.

Having now described the features, discoveries and principles of the disclosure, the manner in which the disclosure is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the disclosure herein described, and all statements of the scope of the disclosure which, as a matter of language, might be said to fall therebetween.

### EXAMPLES

The present disclosure furthermore relates to the following aspects.

Example 1. A guidance device comprising: a first panel, the first panel including: an attachment element on at least a portion of a bottom surface of the first panel, one or more first engagement elements located on a top surface of the first panel, and one or more friction members disposed along the first panel; a second panel movably connected to the first panel via one or more hinges, the second panel including: a guidance element positioned along a the second panel for receiving a medical device, and one or more second engagement elements for engagement with the one or more first engagement elements of the first panel, wherein the first panel and the second panel define a target region, when aligned, and wherein the guidance device is configurable between an unlocked configuration, where the first panel and the second panel are movable relative to each other, and a locked configuration, where the first panel and the second panel are secured relative to each other by engagement of the one or more first engagement elements with the one or more second engagement elements.

Example 2. The guidance device of example 1, wherein the attachment element comprises a dual-sided adhesive, wherein a first side of the dual-sided adhesive is adhered to the bottom surface of the first panel and a second side of the dual-sided adhesive is configured to adhere to an external surface.

Example 3. The guidance device of any one of examples 1-2, wherein each of the one or more friction members is resiliently deformable and aligns with cutouts in the guidance element of the second panel in the locked configuration to restrict movement of at least a portion of the medical device.

Example 4. The guidance device of any one of examples 1-3, further comprising one or more voids within the first panel, the one or more voids positioned along a midline of the first panel and adjacent to or between the one or more friction members.

Example 5. The guidance device of any one of examples 1-4, wherein the guidance element extends beyond both top and bottom surfaces of the second panel, and includes cutouts for interfacing with the one or more friction members of the first panel in the locked configuration.

Example 6. The guidance device of any one of examples 1-5, wherein the first panel and the second panel are connected by one or more hinges, wherein each of the one or more hinges includes a first portion and a second portion.

Example 7. A method for guiding a medical apparatus using a guidance device, the method comprising: positioning a first panel of the guidance device on a patient's skin, wherein the first panel includes an attachment element, one or more first engagement elements, and one or more friction members; aligning a target region defined by the first panel and a second panel of the guidance device with a desired point of entry on the patient's skin; adhering, by way of the attachment element, the first panel to the patient's skin; inserting a medical device into a guidance element positioned along the second panel, which is movably connected to the first panel via one or more hinges; adjusting an angle of the medical device by rotating the second panel relative to the first panel; inserting at least a portion of the medical device into the patient's skin at the aligned point of entry; and securing the first panel and the second panel in a locked configuration by engaging the one or more first engagement elements of the first panel with one or more second engagement elements of the second panel, wherein in the locked configuration, the one or more friction members of the first panel restrict movement of at least a portion of medical device.

Example 8. The method of example 7, wherein adjusting an angle of the medical device further comprises temporarily locking the second panel in an angled intermediate position.

Example 9. The method of any one of examples 7-8, wherein the medical device includes a needle and a catheter.

Example 10. The method of example 9, further comprising removing the needle from the guidance device prior to securing the first panel and the second panel.

Example 11. The method of any one of examples 7-10, wherein one or more of the first engagement elements or the second engagement elements are configured to uncouple the first panel from the second panel.

Example 12. The method of example 11, further comprising one of disengaging or repositioning the medical device after uncoupling the first panel from the second panel.

Example 13. A medical guidance system, comprising: a first panel configured to attach to a patient's skin and including an attachment element and one or more locking elements; a second panel movably connected to the first panel, comprising a guidance element with an open side and including one or more securing elements protruding in a direction opposing the first panel; and a third panel movably connected to the second panel and configured to move between an open position and a closed position, the third panel comprising one or more receiving elements designed to engage with the one or more securing elements of the second panel when in the closed position, wherein the guidance element of the second panel aligns with a guidance element of the third panel to form a continuous guidance pathway for a medical apparatus when the third panel is in the closed position, and wherein the continuous guidance pathway is accessible for insertion, swapping, or removal of the medical apparatus when the third panel is in the open position.

Example 14. The medical guidance system of example 13, wherein the attachment element comprises a dual-sided adhesive, wherein a first side of the dual-sided adhesive is adhered to a bottom surface of the first panel and a second side of the dual-sided adhesive is configured to adhere to an external surface.

Example 15. A method for guiding a medical apparatus using a guidance device, the method comprising: positioning a first panel of the guidance device on a patient's skin, wherein the first panel includes an attachment element, one or more first engagement elements, and one or more friction members; inserting a medical device into a guidance element positioned along the second panel, which is movably connected to the first panel via one or more hinges; inserting at least a portion of the medical device into the patient's skin at an aligned point of entry; inserting a guide wire through the medical device; removing the medical device form the guide wire while the guide wire remains inserted past the patient's skin; inserting a sheath over the guide wire; and securing the first panel and the second panel in a locked configuration by engaging the one or more first engagement elements of the first panel with one or more second engagement elements of the second panel, wherein in the locked configuration, the one or more friction members of the first panel restrict movement of at least a portion of medical device.

Example 16. The method of example 15, wherein adjusting an angle of the medical device further comprises temporarily locking the second panel in an angled intermediate position.

Example 17. The method of any one of examples 15-16, wherein the medical device includes at least one of a needle or a catheter.

Example 18. A guidance device comprising: a fastener; a panel including: a first surface; a second surface opposite the first surface; and a plurality of holes formed on the first surface and configured to receive the fastener, wherein the plurality of holes comprise: a first hole; and a second hole, wherein the fastener is configured to pass through the first hole and the second hole, and form a loop, wherein the loop formed by the fastener is configured to receive a medical device between the fastener and the first surface of the panel, wherein a size of the loop is adjustable to receive the medical device and secure the medical device.

Example 19. The guidance device of example 18, further comprising an attachment element on the second surface of the panel.

Example 20. The guidance device of any one of examples 18-19, further comprising an attachment element on the second surface of the panel, wherein the attachment element comprises a dual-sided adhesive, wherein a first side of the dual-sided adhesive is adhered to the second surface of the panel and a second side of the dual-sided adhesive is configured to adhere to an external surface.

Example 21. The guidance device of any one of examples 18-20, wherein the second surface comprises an adhesive surface configured to adhere to an external surface.

Example 22. The guidance device of any one of examples 18-21, wherein the fastener comprises a wire, a suture, string, or strand.

Example 23. The guidance device of any one of examples 18-22, wherein the loop is formed by the fastener being in a knotted configuration.

Example 24. The guidance device of any one of examples 18-23, wherein the loop is formed by the fastener being in a knotted configuration, wherein the knotted configuration is at least one of a slip knot, an overhand knot, a bowline knot, a figure eight knot, or a taut-line hitch.

Example 25. The guidance device of any one of examples 18-24, wherein the size of the loop is decreased by pulling a free end portion of the fastener away from the loop.

Example 26. The guidance device of any one of examples 18-25, wherein the size of the loop is increased by pulling a portion of the fastener forming the loop away from a free end portion.

Example 27. The guidance device of any one of examples 18-26, further comprising an angle support disposed on the panel, wherein the angle support includes: a first angle surface configured to face the medical device; and a second angle surface opposite the first angle surface and configured to face the first surface of the panel, wherein a first angle formed between the first angle surface and the second angle surface is greater than 0⁰ and less than 90°.

Example 28. The guidance device of any one of examples 27, wherein the panel comprises a slope portion, wherein a second angle formed between the slope portion of the panel and the second angle surface is substantially the same as the first angle formed between the first angle surface and the second angle surface.

Example 29. The guidance device of any one of examples 18-28, wherein the first surface of the panel and the second surface of the panel are substantially parallel to each other.

Example 30. The guidance device of any one of examples 18-29, wherein an angle formed between the first surface of the panel and the second surface of the panel is greater than 0° and less than 90°.

Example 31. The guidance device of any one of examples 18-30, wherein the medical device comprises at least one of a needle or a catheter.

Example 32. A guidance device comprising: a plurality of fasteners; a panel including: a first surface; a second surface opposite the first surface; and a plurality of holes formed on the first surface and configured to receive the plurality of fasteners, wherein the plurality of holes comprise: a first hole; a second hole; a third hole; and a fourth hole; wherein the plurality of fasteners comprise: a first fastener configured to pass through the first hole and the second hole, and form a first loop, a second fastener configured to pass through the third hole and the fourth hole, and form a second loop, wherein the first loop formed by the first fastener is configured to receive a first device, wherein the second loop formed by the second fastener is configured to receive the first device, wherein a size of the first loop and a size of the second loop are adjustable to receive the first device and secure the first device.

Example 33. The guidance device of example 32, wherein the first device is a medical device

Example 34. The guidance device of any one of examples 32-33, wherein the first device is a medical device, wherein the medical device comprises at least one of a needle or a catheter.

Example 35. The guidance device of any one of examples 32-34, wherein the first device is a guidance element configured to guide a medical device.

Example 36. The guidance device of any one of examples 32-35, wherein the first device is a guidance element configured to guide a medical device, wherein the guidance element comprises: a first wing; a second wing; and a guiding portion disposed between the first wing and the second wing and configured to receive a medical device, wherein the first wing is configured to be secured to the panel by the first loop, and the second wing is configured to be secured to the panel by the second loop.

Example 37. The guidance device of any one of examples 32-36, wherein an angle formed between the first surface of the panel and the second surface of the panel is greater than 0° and less than 90°.

What is claimed is:

1. A guidance device comprising:
a fastener;
a panel including:
a first surface;
a second surface opposite the first surface; and
a plurality of holes formed on the first surface and configured to receive the fastener, wherein the plurality of holes comprise:
a first hole; and
a second hole,
wherein the fastener is configured to pass through the first hole and the second hole, and form a loop,
wherein the loop formed by the fastener is configured to receive a medical device between the fastener and the first surface of the panel,
wherein a size of the loop is adjustable to receive the medical device and secure the medical device.

2. The guidance device of claim 1, further comprising an attachment element on the second surface of the panel.

3. The guidance device of claim 1, further comprising an attachment element on the second surface of the panel, wherein the attachment element comprises a dual-sided adhesive, wherein a first side of the dual-sided adhesive is adhered to the second surface of the panel and a second side of the dual-sided adhesive is configured to adhere to an external surface.

4. The guidance device of claim 1, wherein the second surface comprises an adhesive surface configured to adhere to an external surface.

5. The guidance device of claim 1, wherein the fastener comprises a wire, a suture, string, or strand.

6. The guidance device of claim 1, wherein the loop is formed by the fastener being in a knotted configuration.

7. The guidance device of claim 1, wherein the loop is formed by the fastener being in a knotted configuration, wherein the knotted configuration is at least one of a slip knot, an overhand knot, a bowline knot, a figure eight knot, or a taut-line hitch.

8. The guidance device of claim 1, wherein the size of the loop is decreased by pulling a free end portion of the fastener away from the loop.

9. The guidance device of claim 1, wherein the size of the loop is increased by pulling a portion of the fastener forming the loop away from a free end portion.

10. The guidance device of claim 1, further comprising an angle support disposed on the panel, wherein the angle support includes:
a first angle surface configured to face the medical device; and
a second angle surface opposite the first angle surface and configured to face the first surface of the panel,
wherein a first angle formed between the first angle surface and the second angle surface is greater than 0° and less than 90°.

11. The guidance device of claim 10, wherein the panel comprises a slope portion, wherein a second angle formed between the slope portion of the panel and the second angle surface is substantially the same as the first angle formed between the first angle surface and the second angle surface.

12. The guidance device of claim 1, wherein the first surface of the panel and the second surface of the panel are substantially parallel to each other.

13. The guidance device of claim 1, wherein an angle formed between the first surface of the panel and the second surface of the panel is greater than 0° and less than 90°.

14. The guidance device of claim 1, wherein the medical device comprises at least one of a needle or a catheter.

15. A guidance device comprising:
a first panel, the first panel including:
an attachment element on at least a portion of a bottom surface of the first panel,
one or more first engagement elements located on a top surface of the first panel, and
one or more friction members disposed along the first panel;
a second panel movably connected to the first panel via one or more hinges, the second panel including:
a guidance element positioned along a the second panel for receiving a medical device, and
one or more second engagement elements for engagement with the one or more first engagement elements of the first panel,
wherein the first panel and the second panel define a target region, when aligned, and
wherein the guidance device is configurable between an unlocked configuration, where the first panel and the second panel are movable relative to each other, and a locked configuration, where the first panel and the second panel are secured relative to each other by engagement of the one or more first engagement elements with the one or more second engagement elements.

16. The guidance device of claim 15, wherein the attachment element comprises a dual-sided adhesive, wherein a first side of the dual-sided adhesive is adhered to the bottom surface of the first panel and a second side of the dual-sided adhesive is configured to adhere to an external surface.

17. The guidance device of claim 15, wherein each of the one or more friction members is resiliently deformable and aligns with cutouts in the guidance element of the second panel in the locked configuration to restrict movement of at least a portion of the medical device.

18. The guidance device of claim 15, further comprising one or more voids within the first panel, the one or more voids positioned along a midline of the first panel and adjacent to or between the one or more friction members.

19. The guidance device of claim 15, wherein the guidance element extends beyond both top and bottom surfaces of the second panel, and includes cutouts for interfacing with the one or more friction members of the first panel in the locked configuration.

20. The guidance device of claim 15, wherein the first panel and the second panel are connected by one or more hinges, wherein each of the one or more hinges includes a first portion and a second portion.
